SEQUENCE LISTING (84 sequences)

SEQ ID NO: 1 - DNA, 54 bp, Artificial Sequence
  Synthetic oligonucleotide - DnaJNcoF
tgataaccatggaagattctacggttaacacaatggctaagcaagattattacg

SEQ ID NO: 2 - DNA, 30 bp, Artificial Sequence
  Synthetic oligonucleotide - DnaJXhoR
atttcactcgaggcgggtcaggtcgtcaaa

SEQ ID NO: 3 - DNA, 94 bp, Artificial Sequence
  Synthetic oligonucleotide - KHNcoF1
taatgaccatggaaaccgacggttctaaagacgttgttgaaatcgctgttccggaaaacctggttggtgctatcctgggtaaaggtggtaaaac

SEQ ID NO: 4 - DNA, 92 bp, Artificial Sequence
  Synthetic oligonucleotide - KHRecR1
gcccggaacaaattcaccttttttagaaatctggatacgagcacctgtcagttcctggtattcaaccagggttttaccacctttacccagga

SEQ ID NO: 5 - DNA, 92 bp, Artificial Sequence
  Synthetic oligonucleotide - KHRecF2
aaaaggtgaatttgttccgggcacccgtaaccgtaaagttaccatcacaggcaccccggctgctacccaggctgctcagtacctgatcacac

SEQ ID NO: 6 - DNA, 90 bp, Artificial Sequence
  Synthetic oligonucleotide - KHXhoR2
ttatcactcgagttaaccaactttctgcgggttagcagcacgaacaccctgttcgtaggtgatacgctgtgtgatcaggtactgagcagc

SEQ ID NO: 7 - DNA, 48 bp, Artificial Sequence
  Synthetic oligonucleotide - DnaJLinkR
gctgccgccaccaccgctaccgccaccgccgcgggtcaggtcgtcaaa

SEQ ID NO: 8 - DNA, 42 bp, Artificial Sequence
  Synthetic oligonucleotide - LinkKHF
ggtagcggtggtggcggcagcaccgacggttctaaagacgtt

SEQ ID NO: 9 - DNA, 33 bp, Artificial Sequence
  Synthetic oligonucleotide - KHXhoR
atttcactcgagttaaccaactttctgcgggtt

SEQ ID NO: 10 - DNA, 77 bp, Artificial Sequence
  Synthetic oligonucleotide - KH-6xHis-EcoR
cgattaggatcctcatcattaatgatggtggtgatggtgagatccacgcggaaccagaccaactttctgcgggttag

SEQ ID NO: 11 - DNA, 51 bp, Artificial Sequence
  Synthetic oligonucleotide - DnaJR
agaacctccgccgccagaacccccgccaccgcgggtcaggtcgtcaaaaaa

SEQ ID NO: 12 - DNA, 43 bp, Artificial Sequence
  Synthetic oligonucleotide - DnaKF
tctggcggcggaggttctggtaaaataattggtatcgacctgg

SEQ ID NO: 13 - DNA, 42 bp, Artificial Sequence
  Synthetic oligonucleotide - DnaKR
gctaccgccaccgccttttttgtctttgacttcttcaaattc

SEQ ID NO: 14 - DNA, 24 bp, Artificial Sequence
  Synthetic oligonucleotide - KHF
gacaaaaaaggcggtggcggtagc

SEQ ID NO: 15 - DNA, 28 bp, Artificial Sequence
  Synthetic oligonucleotide - ScFvNdeF
tgataacatatgcaggtccaactgcagc

SEQ ID NO: 16 - DNA, 59 bp, Artificial Sequence
  Synthetic oligonucleotide - ScFvXhoR
tcattactcgagtcatcattagtggtggtggtggtggtgtttgatctccagcttggtcc

SEQ ID NO: 17 - DNA, 96 bp, Artificial Sequence
  Synthetic oligonucleotide - ScFv1LXhoR
ccgttactcgagccgcgcggggtgatctaggtccgcgcggtcgtcgtcgtcatcattagtggtggtggtggtggtgtttgatctccagcttggtcc

SEQ ID NO: 18 - DNA, 80 bp, Artificial Sequence
  Synthetic oligonucleotide - 3L-1R
aggtgagcaacggacatccttcacgggtgatctaggtcgtgaaggctcgatcgtcatcattagtggtggtggtggtggtg

SEQ ID NO: 19 - DNA, 87 bp, Artificial Sequence
  Synthetic oligonucleotide - 3LXhoR
ccgttactcgagtcgtagagcggtgatctaggtgctctacggactgcgttgctcggtgatctaggtgagcaacggacatccttcacg

SEQ ID NO: 20 - DNA, 28 bp, Artificial Sequence
  Synthetic oligonucleotide - BR2ScFvNdeF
tgataacatatgcgtgctggtctgcagt

SEQ ID NO: 21 - DNA, 28 bp, Artificial Sequence
  Synthetic oligonucleotide - BR2ScFvNdeF
tgataacatatgcgtgctggtctgcagt

SEQ ID NO: 22 - DNA, 29 bp, Artificial Sequence
  Synthetic oligonucleotide - UGDNdeF
tgataacatatgaaaatcaccatttccgg

SEQ ID NO: 23 - DNA, 55 bp, Artificial Sequence
  Synthetic oligonucleotide - UGDXhoR
ccgttactcgagttattagtggtggtggtggtggtggtcgctgccaaagagatcg

SEQ ID NO: 24 - DNA, 95 bp, Artificial Sequence
  Synthetic oligonucleotide - UGD1LXhoR
ccgttactcgagccgcgcggggtgatctaggtccgcgcggtcgtcgtcgtcatcattagtggtggtggtggtggtggtcgctgccaaagagatcg

SEQ ID NO: 25 - DNA, 32 bp, Artificial Sequence
  Synthetic oligonucleotide - AdhNdeF
tgataacatatgtctatcccagaaactcaaaa

SEQ ID NO: 26 - DNA, 60 bp, Artificial Sequence
  Synthetic oligonucleotide - AdhXhoR
ccgttactcgagttattagtggtggtggtggtggtgtttagaagtgtcaacaacgtatct

SEQ ID NO: 27 - DNA, 100 bp, Artificial Sequence
  Synthetic oligonucleotide - Adh1LXhoR
ccgttactcgagccgcgcggggtgatctaggtccgcgcggtcgtcgtcgtcatcattagtggtggtggtggtggtgtttagaagtgtcaacaacgtatct

SEQ ID NO: 28 - DNA, 80 bp, Artificial Sequence; identical to SEQ ID NO: 18
  Synthetic oligonucleotide - 3L-1R

SEQ ID NO: 29 - DNA, 87 bp, Artificial Sequence; identical to SEQ ID NO: 19
  Synthetic oligonucleotide - 3LXhoR

SEQ ID NO: 30 - DNA, 33 bp, Artificial Sequence
  Synthetic oligonucleotide - UbiCNdeF
tgataacatatgcgattgttgcgtttttgttgc

SEQ ID NO: 31 - DNA, 56 bp, Artificial Sequence
  Synthetic oligonucleotide - UbiCXhoR
ccgttactcgagttattagtggtggtggtggtggtggtacaacggtgacgccggta

SEQ ID NO: 32 - DNA, 96 bp, Artificial Sequence
  Synthetic oligonucleotide - UbiC1LXhoR
ccgttactcgagccgcgcggggtgatctaggtccgcgcggtcgtcgtcgtcatcattagtggtggtggtggtggtggtacaacggtgacgccggta

SEQ ID NO: 33 - DNA, 80 bp, Artificial Sequence; identical to SEQ ID NO: 18
  Synthetic oligonucleotide - 3L-1R

SEQ ID NO: 34 - DNA, 87 bp, Artificial Sequence; identical to SEQ ID NO: 19
  Synthetic oligonucleotide - 3LXhoR

SEQ ID NO: 35 - DNA, 83 bp, Artificial Sequence
  Synthetic oligonucleotide - HIVPrXbaF
attctaaatctagattattcactacgcgttaaggaggtacgacatgcaccatcaccaccatcatcctcaaatcaccctgtggc

SEQ ID NO: 36 - DNA, 85 bp, Artificial Sequence
  Synthetic oligonucleotide - HIVPrXhoR
ccgaattactcgagtcatcattagaagttcagggtgcaaccgatctgggtcagcatgttacgaccgatgatgttgatcggggtcg

SEQ ID NO: 37 - DNA, 77 bp, Artificial Sequence
  Synthetic oligonucleotide - HIVPr1LXhoR
ccgttactcgagccgcgcggggtgatctaggtccgcgcggtcgtcgtcgtcatcattagaagttcagggtgcaaccg

SEQ ID NO: 38 - DNA, 78 bp, Artificial Sequence
  Synthetic oligonucleotide - HIVPrXho3LR
ccgaattactcgagccgcgcggggtgatctaggtccgcgcggtcgtcgtcgtcatcattagaagttcagggtgcaacc

SEQ ID NO: 39 - DNA, 33 bp, Artificial Sequence; identical to SEQ ID NO: 30
  Synthetic oligonucleotide - LepNdeF

SEQ ID NO: 40 - DNA, 56 bp, Artificial Sequence; identical to SEQ ID NO: 31
  Synthetic oligonucleotide - LepXhoR

SEQ ID NO: 41 - DNA, 96 bp, Artificial Sequence; identical to SEQ ID NO: 32
  Synthetic oligonucleotide - Lep1LXhoR

SEQ ID NO: 42 - DNA, 80 bp, Artificial Sequence; identical to SEQ ID NO: 18
  Synthetic oligonucleotide - 3L-1R

SEQ ID NO: 43 - DNA, 87 bp, Artificial Sequence; identical to SEQ ID NO: 19
  Synthetic oligonucleotide - 3LXhoR

SEQ ID NO: 44 - DNA, 30 bp, Artificial Sequence
  Synthetic oligonucleotide - BMP2NdeF
tgataacatatgcaagccaaacacaaacag

SEQ ID NO: 45 - DNA, 54 bp, Artificial Sequence
  Synthetic oligonucleotide - BMP2XhoR
ccgttactcgagttattagtggtggtggtggtggtggcgacacccacaaccctc

SEQ ID NO: 46 - DNA, 93 bp, Artificial Sequence
  Synthetic oligonucleotide - BMP21LXhoR
ccgttactcgagccgcgcggggtgatctaggtccgcgcggtcgtcgtcgtcatcattagtggtggtggtggtggtgcgacacccacaaccctc

SEQ ID NO: 47 - DNA, 80 bp, Artificial Sequence; identical to SEQ ID NO: 18
  Synthetic oligonucleotide - 3L-1R

SEQ ID NO: 48 - DNA, 30 bp, Artificial Sequence; identical to SEQ ID NO: 44
  Synthetic oligonucleotide - sfGFPNdeF

SEQ ID NO: 49 - DNA, 40 bp, Artificial Sequence
  Synthetic oligonucleotide - sfGFPEcoR
aggtcagaattctcatcattacgtaatacctgccgcattc

SEQ ID NO: 50 - DNA, 68 bp, Artificial Sequence
  Synthetic oligonucleotide - CsfGFPXbaF
ccatgatctagaaataattttgtttaactttaagaaggagatataccatggtgcccatccaaaaagtc

SEQ ID NO: 51 - DNA, 42 bp, Artificial Sequence
  Synthetic oligonucleotide - CsfGFPNdeR
ccgttacatatgtcatcattatgtaatcccagcagcatttac

SEQ ID NO: 52 - DNA, 30 bp, Artificial Sequence; identical to SEQ ID NO: 44
  Synthetic oligonucleotide - sfGFPNdeF

SEQ ID NO: 53 - DNA, 44 bp, Artificial Sequence
  Synthetic oligonucleotide - sfNGFPEcoR
aggtcagaattctcatcattatttttcgttcggatctttagaca

SEQ ID NO: 54 - DNA, 85 bp, Artificial Sequence
  Synthetic oligonucleotide - sfNGFP3L-1R
aggtgagcaacggacatccttcacgggtgatctaggtcgtgaaggctcgatcgtcatcattatttttcgttcggatctttagaca

SEQ ID NO: 55 - DNA, 86 bp, Artificial Sequence
  Synthetic oligonucleotide - sfNGFP3LEcoR
aggtcagaattctcgtagagcggtgatctaggtgctctacggactgcgttgctcggtgatctaggtgagcaacggacatccttcac

SEQ ID NO: 56 - DNA, 31 bp, Artificial Sequence
  Synthetic oligonucleotide - DnaJNdeF
tgataacatatggctaagcaagattattacg

SEQ ID NO: 57 - DNA, 55 bp, Artificial Sequence
  Synthetic oligonucleotide - ScFvFpol
gttttttgacgacctgacccgcgaggaggtggaataatgcaggtccaactgcagc

SEQ ID NO: 58 - DNA, 54 bp, Artificial Sequence
  Synthetic oligonucleotide - DnaJFpol
caccaccaccaccaccacgaggaggtggaataatggctaagcaagattattacg

SEQ ID NO: 59 - DNA, 60 bp, Artificial Sequence
  Synthetic oligonucleotide - UbiCFpol
gttttttgacgacctgacccgcgaggaggtggaataatgcgattgttgcgtttttgttgc

SEQ ID NO: 60 - DNA, 76 bp, Artificial Sequence
  Synthetic oligonucleotide - HIVPrFpol
gttttttgacgacctgacccgcgaggaggtggaataatgcaccatcaccaccatcatcctcaaatcaccctgtggc

SEQ ID NO: 61 - DNA, 55 bp, Artificial Sequence
  Synthetic oligonucleotide - DnaJFpolHIVPr
cggttgcaccctgaacttcgaggaggtggaataatggctaagcaagattattacg

SEQ ID NO: 62 - DNA, 60 bp, Artificial Sequence; identical to SEQ ID NO: 59
  Synthetic oligonucleotide - LepFpol

SEQ ID NO: 63 - DNA, 57 bp, Artificial Sequence
  Synthetic oligonucleotide - BMP2FPol
gttttttgacgacctgacccgcgaggaggtggaataatgcaagccaaacacaaacag

SEQ ID NO: 64 - DNA, 59 bp, Artificial Sequence
  Synthetic oligonucleotide - AdhFPol
gttttttgacgacctgacccgcgaggaggtggaataatgtctatcccagaaactcaaaa

SEQ ID NO: 65 - DNA, 43 bp, Artificial Sequence
  Synthetic oligonucleotide - TliANdeF
tgataacatatgcatcatcatcatcatcatcatcatcacagca

SEQ ID NO: 66 - DNA, 42 bp, Artificial Sequence
  Synthetic oligonucleotide - TliAEcoR
ctgagaattctcatcattaactgatcagcacaccctcgctcc

SEQ ID NO: 67 - DNA, 70 bp, Artificial Sequence
  Synthetic oligonucleotide - TliAFPol
gttttttgacgacctgacccgcgaggaggtggaataatgcatcatcatcatcatcatcatcatcacagca

SEQ ID NO: 68 - DNA, 59 bp, Artificial Sequence
  Synthetic oligonucleotide - DnaJTliAFpol
ggagcgagggtgtgctgatcagtgaggaggtggaataatggctaagcaagattattacg

SEQ ID NO: 69 - DNA, 34 bp, Artificial Sequence
  Synthetic oligonucleotide - DnaJEcoR
ctgagaattcttattagcgggtcaggtcgtcaaa

SEQ ID NO: 70 - DNA, 39 bp, Artificial Sequence
  Synthetic oligonucleotide - TliA1EcoR
ccgttagaattctcatcattagtcggtggtcgactcgtg

SEQ ID NO: 71 - DNA, 71 bp, Artificial Sequence
  Synthetic oligonucleotide - TliA1FPol
agttttttgacgacctgacccgcaccggaggtacataatgcatcatcatcatcatcatcatcatcacagca

SEQ ID NO: 72 - DNA, 39 bp, Artificial Sequence; identical to SEQ ID NO: 70
  Synthetic oligonucleotide - TliA1EcoR

SEQ ID NO: 73 - DNA, 31 bp, Artificial Sequence
  Synthetic oligonucleotide - TliA2NdeF
tgataacatatgaacatcgtcagcttcaacg

SEQ ID NO: 74 - DNA, 1131 bp, Escherichia coli
atggctaagcaagattattacgagattttaggcgtttccaaaacagcggaagagcgtgaaatcagaaaggcctacaaacgcctggccatgaaataccacccggaccgtaaccagggtgacaaagaggccgaggcgaaatttaaagagatcaaggaagcttatgaagttctgaccgactcgcaaaaacgtgcggcatacgatcagtatggtcatgctgcgtttgagcaaggtggcatgggcggcggcggttttggcggcggcgcagacttcagcgatatttttggtgacgttttcggcgatatttttggcggcggacgtggtcgtcaacgtgcggcgcgcggtgctgatttacgctataacatggagctcaccctcgaagaagctgtacgtggcgtgaccaaagagatccgcattccgactctggaagagtgtgacgtttgccacggtagcggtgcaaaaccaggtacacagccgcagacttgtccgacctgtcatggttctggtcaggtgcagatgcgccagggattcttcgctgtacagcagacctgtccacactgtcagggccgcggtacgctgatcaaagatccgtgcaacaaatgtcatggtcatggtcgtgttgagcgcagcaaaacgctgtccgttaaaatcccggcaggggtggacactggagaccgcatccgtcttgcgggcgaaggtgaagcgggcgagcatggcgcaccggcaggcgatctgtacgttcaggttcaggttaaacagcacccgattttcgagcgtgaaggcaacaacctgtattgcgaagtcccgatcaacttcgctatggcggcgctgggtggcgaaatcgaagtaccgacccttgatggtcgcgtcaaactgaaagtgcctggcgaaacccagaccggtaagctattccgtatgcgcggtaaaggcgtcaagtctgtccgcggtggcgcacagggtgatttgctgtgccgcgttgtcgtcgaaacaccggtaggcctgaacgaaaggcagaaacagctgctgcaagagctgcaagaaagcttcggtggcccaaccggcgagcacaacagcccgcgctcaaagagcttctttgatggtgtgaagaagttttttgacgacctgacccgctaa

SEQ ID NO: 75 - DNA, 1917 bp, Escherichia coli
atgggtaaaataattggtatcgacctgggtactaccaactcttgtgtagcgattatggatggcaccactcctcgcgtgctggagaacgccgaaggcgatcgcaccacgccttctatcattgcctatacccaggatggtgaaactctagttggtcagccggctaaacgtcaggcagtgacgaacccgcaaaacactctgtttgcgattaaacgcctgattggtcgccgcttccaggacgaagaagtacagcgtgatgtttccatcatgccgttcaaaattattgctgctgataacggcgacgcatgggtcgaagttaaaggccagaaaatggcaccgccgcagatttctgctgaagtgctgaaaaaaatgaagaaaaccgctgaagattacctgggtgaaccggtaactgaagctgttatcaccgtaccggcatactttaacgatgctcagcgtcaggcaaccaaagacgcaggccgtatcgctggtctggaagtaaaacgtatcatcaacgaaccgaccgcagctgcgctggcttacggtctggacaaaggcactggcaaccgtactatcgcggtttatgacctgggtggtggtactttcgatatttctattatcgaaatcgacgaagttgacggcgaaaaaaccttcgaagttctggcaaccaacggtgatacccacctggggggtgaagacttcgacagccgtctgatcaactatctggttgaagaattcaagaaagatcagggcattgacctgcgcaacgatccgctggcaatgcagcgcctgaaagaagcggcagaaaaagcgaaaatcgaactgtcttccgctcagcagaccgacgttaacctgccatacatcactgcagacgcgaccggtccgaaacacatgaacatcaaagtgactcgtgcgaaactggaaagcctggttgaagatctggtaaaccgttccattgagccgctgaaagttgcactgcaggacgctggcctgtccgtatctgatatcgacgacgttatcctcgttggtggtcagactcgtatgccaatggttcagaagaaagttgctgagttctttggtaaagagccgcgtaaagacgttaacccggacgaagctgtagcaatcggtgctgctgttcagggtggtgttctgactggtgacgtaaaagacgtactgctgctggacgttaccccgctgtctctgggtatcgaaacgatgggcggtgtgatgacgacgctgatcgcgaaaaacaccactatcccgaccaagcacagccaggtgttctctaccgctgaagacaaccagtctgcggtaaccatccatgtgctgcagggtgaacgtaaacgtgcggctgataacaaatctctgggtcagttcaacctagatggtatcaacccggcaccgcgcggcatgccgcagatcgaagttaccttcgatatcgatgctgacggtatcctgcacgtttccgcgaaagataaaaacagcggtaaagagcagaagatcaccatcaaggcttcttctggtctgaacgaagatgaaatccagaaaatggtacgcgacgcagaagctaacgccgaagctgaccgtaagtttgaagagctggtacagactcgcaaccagggcgaccatctgctgcacagcacccgtaagcaggttgaagaagcaggcgacaaactgccggctgacgacaaaactgctatcgagtctgcgctgactgcactggaaactgctctgaaaggtgaagacaaagccgctatcgaagcgaaaatgcaggaactggcacaggtttcccagaaactgatggaaatcgcccagcagcaacatgcccagcagcagactgccggtgctgatgcttctgcaaacaacgcgaaagatgacgatgttgtcgacgctgaatttgaagaagtcaaagacaaaaaataa

SEQ ID NO: 76 - DNA, 276 bp, Artificial Sequence
  Synthetic oligonucleotide -Nova-1 KH3 RNA       binding domain
accgacggttctaaagacgttgttgaaatcgctgttccggaaaacctggttggtgctatcctgggtaaaggtggtaaaaccctggttgaataccaggaactgacaggtgctcgtatccagatttctaaaaaaggtgaatttgttccgggcacccgtaaccgtaaagttaccatcacaggcaccccggctgctacccaggctgctcagtacctgatcacacagcgtatcacctacgaacagggtgttcgtgctgctaacccgcagaaagttggttaa

SEQ ID NO: 77 - DNA, 12 bp, Artificial Sequence
  Synthetic oligonucleotide - loop having the       binding site for KH RNA binding domain
acctagatcacc

SEQ ID NO: 78 - protein, 4 aa, Artificial Sequence
  Synthetic oligonucleotide - a region introduced      into the C-terminus of first cistron
EEVE

SEQ ID NO: 79 - DNA, 12 bp, Artificial Sequence
  Synthetic oligonucleotide - a region introduced      into the C-terminus of first cistron
gaggaggtggaa

SEQ ID NO: 80 - DNA, 33 bp, Artificial Sequence
  Synthetic oligonucleotide - 1 loop desipn
cgagaccgcgcggaccuagaucaccccgcgcgg

SEQ ID NO: 81 - DNA, 115 bp, Artificial Sequence
  Synthetic oligonucleotide - 3 loop desipn
cgaucgagccuucacgaccuagaucacccgugaaggauguccguugcucaccuagaucaccgagcaacgcaguccguagagcaccuagaucaccgcucuacgacucgaguaacgg

SEQ ID NO: 82 - DNA, 26 bp, Artificial Sequence
  Synthetic oligonucleotide - Overlapping       two-cistron
  misc_feature (18)..(26): n is a, c, g, or t
gaggaggtggaataatgnnnnnnnnn

SEQ ID NO: 83 - DNA, 26 bp, Artificial Sequence
  Synthetic oligonucleotide - Tandem two-cistron
  misc_feature (19)..(26): n is a, c, g, or t
gaggaggtggaataaatgnnnnnnnn

SEQ ID NO: 84 - DNA, 26 bp, Artificial Sequence
  Synthetic oligonucleotide - n-bp spaced       two-cistron
  misc_feature (16)..(16): n is a, c, g, or t
  misc_feature (20)..(26): n is a, c, g, or t
gaggaggtggaataanatgnnnnnnn

(19) United States
(12) Patent Application Publication
(10) Pub. No.: US 2019/0119688 A1
(43) Pub. Date: Apr. 25, 2019

(54) COMPOSITION FOR SOLUBILIZING TARGET PROTEIN AND USE THEREOF

(71) Applicant: INTELLIGENT SYNTHETIC BIOLOGY CENTER, Daejeon (KR)

(72) Inventors: Jun Hyoung LEE, Daejeon (KR); Sun-Chang KIM, Daejeon (KR); Almando Geraldi, Daejeon (KR)

(21) Appl. No.: 15/884,543

(22) Filed: Jan. 31, 2018

(30) Foreign Application Priority Data
Oct. 25, 2017 (KR) 10-2017-0139497

(51) Int. Cl. C12N 15/62; C07K 14/00

(52) U.S. Cl. CPC C12N 15/625; C07K 14/00; C12N 2310/122; C07K 2319/35; C07K 2319/85; C12N 2310/3519

(57) ABSTRACT

Provided are a composition for solubilizing a target protein, including an expression cassette including a promoter and a gene encoding a fusion protein of a chaperone and an RNA-binding domain or an expression vector including the expression cassette; a transformant including the expression cassette or the expression vector; a kit including the composition or the transformant; and a method for producing the target protein by using the same.

### BACKGROUND OF THE INVENTION

### 1. Field of the Invention

The present invention relates to an expression cassette including a promoter and a gene encoding a fusion protein of a chaperone and an RNA-binding domain; an expression vector including the expression cassette; a composition for solubilizing a target protein, including the expression cassette or the expression vector; a transformant including the expression cassette or the expression vector; a kit including the composition or the transformant; and a method for producing the target protein by using the same.

### 2. Description of the Related Art

When recombinant proteins are produced in heterologous cells, there is a problem that recombinant proteins form insoluble aggregates, and thus there is a need for a method for increasing solubilization of recombinant proteins (Current opinion in biotechnology, 2001, 12, 202-207). However, a method for solubilizing recombinant proteins, which may be applied to various recombinant proteins, has not been developed yet.

Under this background, the present inventors have made many efforts to develop a method for increasing solubilization of a target protein, and as a result, they found that a chaperone-recruiting mRNA scaffold (CRAS) system and a chaperone-substrate co-localized expression (CLEX) system increase solubilization of various target proteins, thereby completing the present invention.

### SUMMARY OF THE INVENTION

An object of the present invention is to provide a composition for solubilizing a target protein, the composition including an expression cassette including a promoter and a gene encoding a fusion protein of a chaperone and an RNA-binding domain or an expression vector including the expression cassette.

Another object of the present invention is to provide a transformant comprising the expression cassette including the promoter and the gene encoding the fusion protein of the chaperone and the RNA-binding domain or the expression vector including the expression cassette.

Still another object of the present invention is to provide a kit for producing the target protein, the kit comprising the composition or the transformant.

Still another object of the present invention is to provide the expression cassette comprising the promoter and the gene encoding the fusion protein of the chaperone and the RNA-binding domain or the expression vector including the expression cassette.

Still another object of the present invention is to provide an expression cassette comprising a promoter, a gene encoding a target protein, and a gene of a hairpin structure at the 3′-terminus thereof, or an expression vector including the expression cassette.

Still another object of the present invention is to provide a method for producing a target protein, the method comprising the steps of:

(i) introducing into a microorganism (a) the expression cassette including the promoter and the gene encoding the fusion protein of the chaperone and the RNA-binding domain or the expression vector including the expression cassette and (b) the expression cassette including the promoter, the gene encoding the target protein, and the gene of the hairpin structure at the 3′-terminus thereof, or the expression vector including the expression cassette; and

(ii) culturing the microorganism.

Still another object of the present invention is to provide an expression cassette comprising (a) a cistron encoding a chaperone and (b) a cistron encoding a target protein, or an expression vector including the expression cassette.

Still another object of the present invention is to provide a composition for solubilizing a target protein, the composition comprising the expression cassette including (a) the cistron encoding the chaperone and (b) the cistron encoding the target protein, or the expression vector including the expression cassette.

Still another object of the present invention is to provide a transformant comprising the expression cassette including (a) the cistron encoding the chaperone and (b) the cistron encoding the target protein, or the expression vector including the expression cassette.

Still another object of the present invention is to provide a kit for producing the target protein, the kit comprising the expression cassette including (a) the cistron encoding the chaperone and (b) the cistron encoding the target protein; the expression vector including the expression cassette; the composition for solubilizing the target protein including the expression cassette or the expression vector; or the transformant including the expression cassette or the expression vector.

Still another object of the present invention is to provide a method for producing the target protein, the method comprising the steps of:

(i) preparing the transformant including the expression cassette including (a) the cistron encoding the chaperone and (b) the cistron encoding the target protein, or the expression vector including the expression cassette; and

(ii) culturing the transformant.

### BRIEF DESCRIPTION OF THE DRAWINGS

FIGS. 1A-F show solubilization of recombinant proteins by a chaperone-recruiting mRNA scaffold (CRAS) system:

(A) is a schematic illustration the CRAS system. A fusion protein of a chaperone molecule (DnaJ, DnaK, or a complex of DnaJ and DnaK (DnaJ-DnaK)) and an RNA-binding domain KH of Nova-1 was expressed together with a target recombinant protein to which a KH cognate stem-loop sequence was inserted as shown in (C). The chaperon complex binds with an initial protein to prevent protein aggregation or transfers the protein to other chaperone molecules for correct folding by introducing the KH-binding structure into the 3′UTR of mRNA;

(B) shows the result of solubilization of eight selected aggregatable recombinant proteins (ScFv, HIV-1 protease, BR2-ScFv, UGD, Adhlp, UbiC, Leptin, BMP2) when they were expressed with DnaJ-KH alone; or DnaJ-KH and 3′UTR hairpin loop;

(D) shows the result of solubilization according to one (+) or three (+++) 3′UTR sequence(s), when four recombinant proteins which were not solubilized by the CRAS system using DnaJ-KH were applied to a CRAS system using a chimeric DnaJ-KH;

(E) illustrates a split GFP assay to examine in vivo protein solubilization activity of the CRAS system; and

(F) shows the result of in vivo solubilization of sfGFP N-terminus by the CRAS system. Fluorescence intensities of E. coli BL21(DE3) cells including (1) pET16b and pAMT7 (a negative control group); (2) pET16b-sfGFP and pAMT7-DnaJK-KH (a positive control group); (3) plasmid of sfGFP N-terminus and C-terminus in the absence of KH-binding hairpin and DnaJ-KH; (4) plasmid of sfGFP N-terminus and C-terminus in the presence of KH-binding hairpin and in the absence of DnaJ-KH; (5) plasmid of sfGFP N-terminus and C-terminus in the absence of KH-binding hairpin and in the presence of DnaJ-KH; or (6) plasmid of sfGFP N-terminus and C-terminus in presence of KH-binding hairpin and DnaJ-KH (GRAS system). Quantification of soluble ScFv was performed on SDS-PAGE images obtained by using an ImageJ v1.48 software (NHI, USA) through Coomassie blue staining. The error bars in (B), (D) and (F) represent ±standard deviations from three independent experiments.

FIGS. 2A-B show solubilization of ScFv over time after applying the CRAS system having one loop (A) or three loops (B) in E. coli BL21(DE3):

lane M, marker (ELPIS, Daejeon, South Korea); lane W, whole cell lysate; lane S, soluble fraction; lane I, insoluble fraction.

FIGS. 3A-3 shows the result of solubilization according to the length of a spacer between the stop codon and the 3′UTR-binding loop in the CRAS system. When the CRAS system was applied (B), ScFv solubility was increased, as compared with single expression of ScFv (A), 4 hours after induction of expression:

lane 1, in the absence of spacer between stop codon and 3′UTR-binding loop; lane 2, in the presence of 5-nt spacer between stop codon and 3′UTR-binding loop; lane 3, in the presence of 30-nt spacer between stop codon and 3′UTR-binding loop; lane M, marker; lane W, whole cell lysate; lane S, soluble fraction; lane I, insoluble fraction.

FIG. 4 shows increased solubility of ScFv by the CRAS system in a dnaK knockout strain (lane C-: whole cell lysate of E. coli BL21(DE3) having pET16b and pAMT7 (a negative control group), lane 1: in the absence of KH-binding hairpin and DnaJ-KH, lane 2: in the presence of KH-binding hairpin and in the absence of DnaJ-KH, lane 3: in the absence of KH-binding hairpin and in the presence of DnaJ-KH, lane 4: in the presence of KH-binding hairpin and DnaJ-KH (RNA Scaffold), lane M: marker, lane W: whole cell lysate, lane S: soluble fraction, lane I: insoluble fraction).

FIGS. 5A-F show solubilization of recombinant proteins by a CLEX system:

(A) is a schematic illustration of the CLEX system. A translationally coupled two-cistron expression system was used to translate DnaJ and target protein in proximity, from a single mRNA transcript;

(B) shows overlapping of the stop codon (TAA) of the first cistron with the start codon (ATG) of the second cistron, or a tandem arrangement thereof, or a space of n nt placed therebetween. 12 nucleotides in the 3′-sequence of the first cistron function as a ribosome-binding site (RBS) for the second cistron;

(C) shows the result of solubilization of five aggregatable recombinant proteins when chaperone DnaJ was used as the first or second cistron in the CLEX system;

(D) shows the result of measuring OD₄₅₀ of samples obtained from E. coli BL2J (DE3) having the following plasmids in order to examine a level of correctly folded leptin: pET16b (a negative control group), pET16b-Leptin (leptin overexpression) and pET16b-DnaJ/Leptin (DnaJ and leptin overexpression in CLEX system, CLEX system);

(E) shows the result of solubilization of BMP2 according to the order of DnaJ and target protein in the translationally coupled two-cistron expression system; and

(F) shows the result of solubilization of BMP2 according to the distance between the cistrons in the translationally coupled two-cistron expression system;

the slash (/) between the proteins represents the order of the genes encoding the respective proteins in mRNA. Relative expression were quantified by using an ImageJ v1.48 software (NJI, USA), based on the result of SDS-PAGE. The error bars in (C), (D), (E), and (F) represent ±standard deviations from three independent experiments.

FIGS. 6A-B show increased solubilization of BMP2 in dnaJ knockout strain (A) or dnaK knockout strain (B) by the CLEX system (lane C-: whole cell lysate of E. coli BL21(DE3) having pET16b and pAMT7 (a negative control group), lane only BMP2: BMP2 expression in the absence of DnaJ, lane BMP2+DnaJ: BMP2 expression in the presence of DnaJ, lane DnaJ/BMP2: DnaJ placed in the first cistron and BMP2 placed in the second cistron in the CLEX system, lane M: marker, lane W: whole cell lysate, lane S: soluble fraction, lane I: insoluble fraction).

FIGS. 7A-B shows the result of solubilization according to the size of recombinant protein in the CLEX system:

(A) shows distribution of a DnaJ/DnaK-binding sequence in lipase TliA, indicated by red rectangular blocks. The binding site was predicted by using a Limbo chaperone binding site predictor; and

(B) shows the result of solubilization of TliA1 fragment by the CLEX system. TliA1+DnaJ represents simple coexpression of TliA and DnaJ, DnaJ/TliA1 represents CLEX system using DnaJ as the first cistron. Relative expression levels were quantified by using an ImageJ v1.48 software (NJI, USA), based on the result of SDS-PAGE. The error bars in (B) represent ±standard deviations from three independent experiments.

### DETAILED DESCRIPTION OF THE PREFERRED EMBODIMENTS

To achieve the above objects, an aspect of the present invention provides a composition for solubilizing a target protein, the composition including an expression cassette including a promoter and a gene encoding a fusion protein of a chaperone and a RNA-binding domain, or an expression vector including the expression cassette.

The fusion protein expressed by the expression cassette or the expression vector binds to mRNA including a sequence of the target protein via the RNA-binding domain, and as a result, the chaperone of the fusion protein exists near the translated target protein, leading to correct folding of the target protein. That is, the fusion protein may increase solubility of the target protein in a general manner, irrespective of the kind of the target protein. In this case, mRNA including the sequence of the target protein may have a hairpin structure (including an RNA site to which the RNA-binding domain of the fusion protein binds) at 3′-UTR in order to bind with the fusion protein.

As used herein, the term “chaperone” refers to a protein involved in folding and unfolding or assembly and disassembly of other proteins. Specifically, the chaperone of the present invention functions to induce or help folding of the target protein, thereby increasing solubility of the target protein. In the present invention, the chaperone, in the form of the fusion protein with the RNA-binding domain, binds to mRNA including the sequence of the target protein to help folding of the target protein near the translated target protein.

In the present invention, the chaperone is not limited to an origin (e.g., derived from a microorganism) and a specific sequence thereof, as long as it is involved in folding of the target protein. Specifically, the chaperone may be a protein derived from prokaryotes (e.g., bacteria, etc.), eukaryotes, or archaea, and a sequence thereof may be obtained from a known database, GenBank or NCBI, etc. Specifically, the chaperone may be DnaJ, Dnak, GroEL, GroES, HtpG, ClpA, ClpX, or ClpP, and more specifically, DnaJ or Dnak. The DnaJ or Dnak may have an amino acid sequence encoded by a nucleotide sequence of SEQ ID NO: 74 or 75, but is not limited thereto. Further, the chaperone may be Hsp40, Hsp60, Hsp70, Hsp90, Hsp100, or Hsp104.

In the present invention, the chaperone may be in a combination of two or more of the same or different kind of chaperone units. Specifically, the chaperone may be DnaJK in which DnaJ and Dnak fuse to each other, but is not limited thereto.

As used herein, the term “RNA-binding domain (RBD)” refers to a domain binding to RNA. Specifically, the RNA-binding domain of the present invention forms a complex or fusion protein, together with the chaperone, such that the complex or fusion protein including the chaperone binds to mRNA including the sequence of the target protein. That is, the RNA-binding domain locates the chaperone of the fusion protein near the target protein to be translated by ribosomes, leading to correct folding of the target protein through the adjacent chaperone.

In the present invention, the RNA-binding domain is not limited to an origin (e.g., derived from a microorganism) and a specific sequence thereof, as long as it binds to mRNA including the target protein. Specifically, the RNA-binding domain may be K Homology (KH) domain, bacteriophage MS2 coat protein, bacteriophage PP7 coat protein, sterile alpha motif (SAM), or RNA recognition motif (RRM), and a sequence thereof may be obtained from a known database, GenBank of NCBI, etc. Further, the KH domain may have an amino acid sequence encoded by a nucleotide sequence of SEQ ID NO: 76, but is not limited thereto.

In the present invention, the fusion protein may include one or more chaperones and the RNA-binding domain. Further, in the fusion protein, the chaperone may be linked to the N-terminus; the C-terminus; or the N-terminus and C-terminus of the RNA-binding domain, directly or via a linker. In the fusion protein, the RNA-binding domain may be linked to the N-terminus; the C-terminus; or the N-terminus and C-terminus of the chaperone, directly or via a linker.

The linker is not particularly limited, as long as it allows the chaperone and the RNA-binding domain of the fusion protein to exhibit activity. Specifically, the chaperone and the RNA-binding domain may be linked by using an amino acid such as glycine, alanine, leucine, isoleucine, proline, serine, threonine, asparagine, aspartic acid, cysteine, glutamine, glutamic acid, lysine, argininic acid, etc., and more specifically, by using several amino acids such as valine, leucine, aspartic acid, glycine, alanine, proline, etc. Much more specifically, for easy genetic manipulation, 1 to 10 amino acids of glycine, valine, leucine, aspartic acid, etc. may be linked and used. For example, in the present invention, the fusion protein was prepared by linking the C-terminus of the chaperone to the N-terminus of the RNA-binding domain via a G4S linker.

The fusion protein may incline a polypeptide having a sequence in which one or more amino acid residues are different from those of an amino acid sequence of the wild-type of each protein or domain included in the fusion protein. Amino acid exchanges in proteins and polypeptides, which do not generally alter the activity of molecules, are known in the art. The most commonly occurring exchanges are Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Gln, Asp/Gly, in both directions. The fusion protein may also include proteins with increased structural stability against heat, pH, etc. or increased activities by mutation or modification of the amino acid sequences.

The fusion protein or the polypeptide constituting the fusion protein may be prepared by a chemical peptide synthetic method known in the art, or prepared by amplifying the gene encoding the fusion protein by polymerase chain reaction (PCR) or synthesizing the gene by a known method and then cloning the gene into an expression vector to express the gene.

In a specific embodiment of the present invention, the fusion protein may have a form (DnaJK-KH), in which DnaJ and Dnak (DnaJK) as the chaperones and the KH domain as the RNA-binding domain bind to each other, but is not limited thereto.

The protein of the present invention may include not only the amino acid sequence encoded by the nucleotide sequence of each SEQ ID NO., but also an amino acid sequence having 80% or more, specifically 90% or more, more specifically 95% or more, much more specifically 97% or more sequence homology with the above sequence. Any amino acid sequence having the homology may be included without limitation, as long as it is an amino acid sequence of a protein having an efficacy that is substantially identical or corresponding to that of the above protein. It is apparent that an amino acid sequence having a deletion, modification, substitution, or addition of some sequence may be also within the scope of the present invention, as long as the amino acid sequence has the homology.

Furthermore, the gene encoding the protein of the present invention may include not only the nucleotide sequence of each SEQ ID NO., but also a nucleotide sequence having 80% or more, specifically 90% or more, more specifically 95% or more, much more specifically 98% or more, and most specifically 99% or more sequence homology with the above sequence, as long as it is a nucleotide sequence encoding a protein having an efficacy that is substantially identical or corresponding to that of the above protein. It is apparent that a nucleotide sequence having a deletion, modification, substitution, or addition of some sequence may be also within the scope of the present invention, as long as the nucleotide sequence has the homology.

As used herein, the term “homology” is intended to indicate the degree of similarity of a nucleotide sequence of a gene encoding a protein or an amino acid sequence, and if homology is sufficiently high, an expression product of the corresponding gene has the same or similar activity.

As used herein, the term “promoter” refers to a nucleotide sequence that regulates expression of another nucleotide sequence operably linked thereto in appropriate host cells, and as used herein, “operably linked” refers to a functional linkage between a nucleotide expression control sequence and a nucleotide sequence encoding a target protein or RNA in such a manner as to allow general functions. For example, a promoter and a nucleotide sequence encoding a protein or RNA are operably linked to influence expression of a coding sequence. A method for operably linking the promoter and the nucleotide sequence encoding the protein or RNA may be performed by a genetic recombinant technique well known in the art, and site-specific DNA cleavage and ligation may be achieved by using enzymes generally known in the art.

As used herein, the term “expression vector” refers to a DNA construct including the expression cassette, which is operably linked to an appropriate regulatory sequence to express the target protein in an appropriate host. The regulatory sequence may include a promoter capable of initiating transcription, an arbitrary operator sequence for regulating transcription, a sequence encoding an appropriate mRNA ribosome binding site, and sequences for regulating the termination of transcription and translation. Once a vector is transformed into an appropriate host, the vector may replicate and function independently of the host genome, or it may be integrated into the genome itself.

As long as the expression vector used in the present invention is replicable in host cells, any vector known in the art may be used without particular limitations. Example of the vector commonly used may include a natural or recombinant plasmid, cosmic, virus, and bacteriophage. Example of the phage vector or the cosmid vector may include pWE15, M13, MBL3, MBL4, IXII, ASHII, APII, t10, t11, Charon4A, Charon21A, etc. The plasmid vector may include pBR type, pUC type, pBluescriptII type, pGEM type, pTZ type, pCL type, pET type, pCP type etc. Specifically, pDZ, pACYC177, pACYC184, pCL, pECCG117, pUC19, pBR322, pMW118, pCClBAC, pCP22, pAMT7, pET16b vector, etc. may be used, but is not limited thereto.

The vector usable in the present application is not particularly limited, and any known expression vector may be used. Also, the polynucleotide encoding the target protein may be inserted into the chromosome by a vector for chromosomal gene insertion. The insertion of the polynucleotide into the chromosome may be achieved by any method known in the art, for example, by homologous recombination, but is not limited thereto. A selection marker for confirming the chromosomal insertion may be further included. The selection marker is used to select a cell transformed with the vector, i.e., to confirm the insertion of the target nucleotide molecule. Markers that provide selectable phenotypes, such as drug resistance, auxotrophy, resistance to cytotoxic agents, or surface protein expression, may be used. Only cells expressing the selection marker are able to survive or to show different phenotypes under the environment treated with the selective agent, and thus the transformed cells may be selected.

The composition of the present invention may further include an expression cassette including a promoter, a gene encoding a target protein, and a gene of a hairpin structure at the 3′-terminus thereof, or an expression vector including the expression cassette. The promoter and the expression vector are the same as described above.

mRNA transcribed by the expression cassette or the expression vector has RNA of the target protein and the hairpin structure at 3′-UTR thereof, and the hairpin and the RNA-binding domain of the fusion protein may bind to each other. By this binding, a scaffold, in which the chaperone region of the fusion protein is recruited to 3′-UTR of mRNA including the sequence of the target protein, is formed. In the present invention, this scaffold is called chaperone-recruiting mRNA scaffold (CRAS).

As used herein, the term “hairpin” means a structure consisting of a double-stranded stem and a loop, which is formed by internal hydrogen bonding between bases of single-stranded DNA or RNA. The hairpin is used interchangeably with stem-loop. A sequence of the hairpin may be any sequence known in the art.

Further, the hairpin structure may have an RNA sequence (site), to which the RNA-binding domain of the fusion protein, i.e., the RNA-binding domain linked to the chaperone binds. Therefore, the gene of the hairpin structure included in the expression cassette or expression vector may include a sequence transcribing the RNA sequence to which the RNA-binding domain binds.

In the present invention, the expression cassette or expression vector may further include a spacer gene between the gene encoding the target protein and the gene of the hairpin structure. The spacer gene refers to a non-coding gene between the genes, and any sequence known in the art may be used.

In the present invention, the gene of the hairpin structure may have one or more of the gene of the hairpin structure. In this case, one or more of the hairpin, to which the fusion protein of the present invention binds, may exist in mRNA including the sequence of the target protein, and therefore, a larger number of chaperones may be recruited to the mRNA.

One more of the of the hairpin structure may include a spacer gene between hairpin structures, but is not limited thereto.

In the present invention, the expression cassette or expression vector may further include a ribosome-binding site. The “ribosome-binding site (RBS)” refers to a ribosome-binding site that allows mRNA transcribed from DNA to bind with ribosomes in host cells during initiation of protein biosynthesis. The ribosome-binding site may be any sequence known in the art.

Another aspect of the present invention provides a transformant including the expression cassette including the promoter and the gene encoding the fusion protein of the chaperone and the RNA-binding domain or the expression vector including the expression cassette. Specifically, the transformant may further include the expression cassette including the promoter, the gene encoding the target protein, and the gene of the hairpin structure at the 3′-terminus thereof, or the expression vector including the expression cassette.

The promoter, the chaperone, the RNA-binding domain, the fusion protein, the gene of the hairpin structure, the expression cassette, and the expression vector are the same as described above.

The transformant may produce the target protein with improved solubility by CRAS system.

As used herein, the term “transformation” means that the expression cassette of the present invention or the expression vector including the expression cassette is introduced into a host cell in such a way that the protein encoded the polynucleotide of the expression cassette or the expression vector is expressed in the host cell. As long as the transformed polynucleotide may be expressed in the host cell, it may be either integrated into and placed in the chromosome of the host cell, or exist extrachromosomally. Further, the polynucleotide may include DNA and RNA encoding the target protein. The polynucleotide may be introduced in any form, as long as it may be introduced into the host cell and expressed therein. For example, the polynucleotide may be introduced into the host cell in the form of an expression cassette, which is a gene construct including all elements required for its autonomous expression. Generally, the expression cassette may include a promoter operably linked to the polynucleotide, transcriptional termination signals, ribosome binding sites, or translation termination signals. The expression cassette may be in the form of a self-replicable expression vector. Also, the polynucleotide as it is may be introduced into the host cell and operably linked to sequences required for expression in the host cell, but is not limited thereto. Any transformation method may be employed, as long as it is used to introduce a nucleotide into a host cell. Depending on the host cell, a suitable standard technique may be selected as known in the art. For example, electroporation, calcium phosphate (CaPO₄) precipitation, calcium chloride (CaCl₂)) precipitation, microinjection, a polyethyleneglycol (PEG) technique, a DEAE-dextran technique, a cationic liposome technique, and a lithium acetate DMSO technique may be used, but is not limited thereto.

The transformant is not limited, as long as the protein encoded by the polynucleotide of the expression cassette or expression vector of the present invention may be expressed therein. The transformant may be specifically a plant cell, a bacterial cell, a fungal cell, a yeast cell, or an animal cell, and more specifically, a microorganism of the genus Escherichia.

Still another aspect provides a kit for producing the target protein, the kit including the composition for solubilizing the target protein or the transformant.

Still another aspect provides the expression cassette including the promoter and the gene encoding the fusion protein of the chaperone and the RNA-binding domain, or the expression vector including the expression cassette.

The promoter, the chaperone, the RNA-binding domain, the fusion protein, the expression cassette, and the expression vector are the same as described above.

Still another aspect provides the expression cassette including the promoter, the gene encoding the target protein, and the gene of the hairpin structure at the 3′-terminus thereof, or the expression vector including the expression cassette.

The promoter, the gene of the hairpin structure, the expression cassette, and the expression vector are the same as described above.

Still another aspect provides a kit for producing the target protein, the kit including the composition or the transformant.

Still another aspect provides a method for producing the target protein, the method including the steps of:

(i) preparing a transformant including (a) the expression cassette including the promoter and the gene encoding the fusion protein of the chaperone and the RNA-binding domain or the expression vector including the expression cassette and (b) the expression cassette including the promoter, the gene encoding the target protein, and the gene of the hairpin structure at the 3′-terminus thereof, or the expression vector including the expression cassette; and

(ii) culturing the transformant.

The promoter, the chaperone, the RNA-binding domain, the fusion protein, the gene of the hairpin structure, the expression cassette, the expression vector, and the transformant are the same as described above.

The step (i) of preparing the transformant may be a step of additionally introducing (a) the expression cassette including the promoter and the gene encoding the fusion protein or the expression vector including the expression cassette into a host cell previously having (b) the expression cassette including the promoter, the gene encoding the target protein, and the gene of the hairpin structure at the 3′-terminus thereof, or the expression vector including the expression cassette, but is not limited thereto.

Further, the step (i) of preparing the transformant may be a step of additionally introducing (b) the expression cassette including the promoter, the gene encoding the target; protein, and the gene of the hairpin structure at the 3′-terminus thereof, or the expression vector including the expression cassette into a host cell previously having (a) the expression cassette including the promoter and the gene encoding the fusion protein or the expression vector including the expression cassette, but is not limited thereto.

Furthermore, the step (i) of preparing the transformant may be a step of introducing into the host cell (a) the expression cassette including the promoter and the gene encoding the fusion protein or the expression vector including the expression cassette and (b) the expression cassette including the promoter, the gene encoding the target protein, and the gene of the hairpin structure at the 3′-terminus thereof, or the expression vector including the expression cassette sequentially, simultaneously, or reversely, but is not limited thereto.

In the production method, the step of (ii) culturing the transformant may be performed by, but is not particularly limited to, a known batch, continuous, or fed-batch culture method. In this regard, culture conditions may be, but are not particularly limited to, maintained at optimal pH (e.g., pH 5 to pH 9, specifically pH 6 to pH 8, and most specifically pH 6.8) by using basic compounds (e.g., sodium hydroxide, potassium hydroxide, or ammonia) or acidic compounds (e.g., phosphoric acid or sulfuric acid) and at an aerobic condition by adding oxygen or oxygen-containing gas mixture to a cell culture. The culture temperature may be maintained at 20° C. to 45° C., and specifically at 25° C. to 40° C., and cultured for about 10 hours to about 160 hours, but is not limited thereto. The target protein produced by the culturing may be secreted to a culture medium or may remain within cells.

Further, the culture medium to be used may include sugars and carbohydrates (e.g., glucose, sucrose, lactose, fructose, maltose, molasses, starch, and cellulose), oils and fats (e.g., soybean oil, sunflower seed oil, peanut oil, and coconut oil), fatty acids (e.g., palmitic acid, stearic acid, and linoleic acid), alcohols (e.g., glycerol and ethanol), and organic acids (e.g., acetic acid) as a carbon source individually or in combination, but is not limited thereto. As a nitrogen source, nitrogen-containing organic compounds (e.g., peptone, yeast extract, beef stock, malt extract, corn steep liquor, soybean meal powder, and urea), or inorganic compounds (e.g., ammonium sulfate, ammonium chloride, ammonium phosphate, ammonium carbonate, and ammonium nitrate) may be used individually or in combination, but is not limited thereto. As a phosphorus source, potassium dihydrogen phosphate, dipotassium phosphate, or sodium-containing salt corresponding thereto may be used individually or in combination, but is not limited thereto. The culture medium may include other essential growth-stimulating substances such as metal salts (e.g., magnesium sulfate or iron sulfate), amino acids, and vitamins.

The production method may further include the step of collecting the target protein from the transformant or the culture thereof. The method for collecting the target protein produced in the culturing step may be performed by an appropriate method known in the art according to the culturing method, thereby collecting the target protein from the transformant or the culture thereof. For example, cell lysis, centrifugation, filtration, anion exchange chromatography, crystallization, HPLC, etc. may be used. The target protein may be collected from the transformant or the culture thereof by an appropriate method known in the art.

Still another aspect provides a method for solubilizing a target protein, the method including the steps of:

(i) preparing a transformant including (a) the expression cassette including the promoter and the gene encoding the fusion protein of the chaperone and the RNA-binding domain or the expression vector including the expression cassette and (b) the expression cassette including the promoter, the gene encoding the target protein, and the gene of the hairpin structure at the 3′-terminus thereof, or the expression vector including the expression cassette; and

(ii) culturing the transformant.

The promoter, the chaperone, the RNA-binding domain, the fusion protein, the gene of the hairpin structure, the expression cassette, the expression vector, the transformant, step (i), and step (ii) are the same as described above.

Still another aspect of the present invention provides an expression cassette including (a) a cistron encoding a chaperone and (b) a cistron encoding a target protein, or an expression vector including the expression cassette.

The chaperone, the expression cassette, and the expression vector are the same as described above.

Both the chaperone and the target protein are translated by the expression cassette or the expression vector, and as a result, the chaperone may increase correct folding of the target protein. In the present invention, the system is called a CLEX (chaperone-substrate co-localized expression) system.

(a) and (b) may be connected in either sequential or reverse order from 5′- to 3′-direction, and the stop codon of (a) or (b) may be overlapped with the start codon of (b) or (a), but is not limited thereto.

Still another aspect of the present invention provides a composition for solubilizing the target protein, the composition including the expression cassette including (a) the cistron encoding the chaperone and (b) the cistron encoding the target protein, or the expression vector including the expression cassette.

The chaperone, the expression cassette, and the expression vector are the same as described above.

Still another aspect of the present invention provides a transformant including the expression cassette including (a) the cistron encoding the chaperone and (b) the cistron encoding the target protein, or the expression vector including the expression cassette.

The chaperone, the expression cassette, the expression vector, and the transformant are the same as described above.

Still another aspect of the present invention provides a kit for producing the target protein, the kit including the expression cassette including (a) the cistron encoding the chaperone and (b) the cistron encoding the target protein; the expression vector including the expression cassette; the composition for solubilizing the target protein including the expression cassette or the expression vector; or the transformant including the expression cassette or the expression vector.

The chaperone, the expression cassette, the expression vector, and the transformant are the same as described above.

Still another aspect of the present invention provides a method for producing the target protein, the method including the steps of:

(i) preparing the transformant including the expression cassette including (a) the cistron encoding the chaperone and (b) the cistron encoding the target protein, or the expression vector including the expression cassette; and

(ii) culturing the transformant.

The chaperone, the expression cassette, the expression vector, the transformant, the culturing, and the production method are the same as described above.

The production method may further include the step of collecting the target protein from the transformant or the culture thereof.

Hereinafter, the construction and effect of the present invention will be described in more detail with reference to Examples. However, these Examples are for illustrative purposes only, and the scope of the present invention is not intended to be limited by these Examples.

### Experimental Example 1. Microorganisms, Enzymes, and Chemicals

E. coli XL1-Blue (Stratagene, La Jolla, Calif., USA) and BL21(DE3) (Novagen, Madison, Wis., USA) strains were used. XL-1 Blue strain was used for general cloning and BL21(DE3) strain was used for gene expression. A strain free of dnaJ and dnaK genes was prepared by known PI transduction (Current protocols in molecular biology, 2007, Chapter 1, Unit1 17). BW25113 strains having single knock-outs (ΔdnaJ and ΔdnaK) were obtained from Keio collection, and used as a donor strain for BL21(DE3). Colony PCR was performed to screen deletion strains by using primer pairs around a target gene (Table 1). Then, a kanamycin resistance cassette was removed from the integration region in the host genome by using a pCP22 plasmid-derived FLP recombinant enzyme. Oligonucleotides (Genotech, Daejeon, Korea) and genes (Bioneer, Daejeon, Korea) used in the present invention are described in Table 1. Chemicals were purchased from Sigma-Aldrich (St. Louis, Mo., USA).

TABLE 1

| | | SEQ | |
|---|---|---|---|
| Proteins/ | | ID | |
| genes | Plasmid | NO. | Primers (5′-3′) |
| DnaJ | pAMT7-DnaJ | 1 | DnaJNcoF: 5′-TGATAACCATGGAAGATTCTACGGTTAACACAATGG |
| (NcoI/XhoI) | (PT7::dnaJ; | | CTAAGCAAGATTATTACG-3′ |
| | pBR322 ori, | 2 | DnaJXhoR: 5′-ATTTCACTCGAGGCGGGTCAGGTCGTCAAA-3′ |
| | Cm^{R}) | | |
| KH RNA | pAMT7-KH | 3 | KHNcoF1: 5′-TAATGACCATGGAAACCGACGGTTCTAAAGACGTTGT |
| binding | (PT7::KH; | | TGAAATCGCTGTTCCGGAAAACCTGGTTGGTGCTATCCTGGGTAA |
| domain | pBR322 ori, | | AGGTGGTAAAAC-3′ |
| (NcoI/XhoI) | Cm^{R}) | 4 | KHRecR1: 5′-GCCCGGAACAAATTCACCTTTTTTAGAAATCTGGATA |
| | | | CGAGCACCTGTCAGTTCCTGGTATTCAACCAGGGTTTTACCACCTT |
| | | | TACCCAGGA-3′ |
| | | 5 | KHRecF2: 5′-AAAAGGTGAATTTGTTCCGGGCACCCGTAACCGTAA |
| | | | AGTTACCATCACAGGCACCCCGGCTGCTACCCAGGCTGCTCAGTA |
| | | | CCTGATCACAC-3′ |
| | | 6 | KHXhoR2: 5′-TTATCACTCGAGTTAACCAACTTTCTGCGGGTTAGCA |
| | | | GCACGAACACCCTGTTCGTAGGTGATACGCTGTGTGATCAGGTAC |
| | | | TGAGCAGC-3′ |
| DnaJ-KH | pAMT7- | 1 | DnaJNcoF: 5′-TGATAACCATGGAAGATTCTACGGTTAACACAAT |
| (NcoI/XhoI) | dnaJ-KH | | GGCTAAGCAAGATTATTACG-3′ |
| | (PT7:: | 7 | DnaJLinkR: 5′-GCTGCCGCCACCACCGCTACCGCCACCGCCGCG |
| | dnaJ-KH; | | GGTCAGGTCGTCAAA-3′ |
| | pBR322 ori, | 8 | LinkKHF: 5′-GGTAGCGGTGGTGGCGGCAGCACCGACGGTTCTAAA |
| | Cm^{R}) | | GACGTT-3′ |
| | | 9 | KHXhoR: 5′-ATTTCACTCGAGTTAACCAACTTTCTGCGGGTT-3′ |
| DnaJ-KH- | pAMT7- | 1 | DnaJNcoF: 5′-TGATAACCATGGAAGATTCTACGGTTAACACAAT |
| 6xHis | DnaJ-KH- | | GGCTAAGCAAGATTATTACG-3′ |
| (NcoI/EcoRI) | 6xHis | 10 | KH-6xHis-EcoR: 5′-CGATTAGGATCCTCATCATTAATGATGGTGGTG |
| | (PT7::dnaJ- | | ATGGTGAGATCCACGCGGAACCAGACCAACTTTCTGCGGGTTAG-3′ |
| | KH; pBR322 | | |
| | ori, Cm^{R}) | | |
| DnaJK-KH | pAMT7- | 1 | DaJNcoF: 5′-TGATAACCATGGAAGATTCTACGGTTAACACAAT |
| (NcoI/XhoI) | DnaJK-KH | | GGCTAAGCAAGATTATTACG-3′ |
| | (PT7::dnaJ- | 11 | DnaJR: 5′-AGAACCTCCGCCGCCAGAACCCCCGCCACCGCGGGTC |
| | dnaK-KH; | | AGGTCGTCAAAAAA-3′ |
| | pBR322 ori, | 12 | DnaKF: 5′-_TCTGGCGGCGGAGGTTCTGGTAAAATAATTGGTATCGA |
| | Cm^{R}) | | CCTGG-3′ |
| | | 13 | DnaKR: 5′-_GCTACCGCCACCGCCTTTTTTGTCTTTGACTTCTTCAA |
| | | | TTC-3′ |
| | | 14 | KHF: 5′-GACAAAAAAGGCGGTGGCGGTAGC-3′ |
| | | 9 | KHXhoR: 5′-TTTCCAGAACTCGAGTTAACCAACTTTCTGCGGGT-3′ |
| ScFv | pET16b- | 15 | ScFvNdeF: 5′-TGATAACATATGCAGGTCCAACTGCAGC-3′ |
| (NdeI/XhoI) | ScFv | 16 | ScFvXhoR: 5′-TCATTACTCGAGTCATCATTAGTGGTGGTGGTGGTG |
| | (PT7::scFv; | | GRGTTTGATCTCCAGCTTGGTCC-3′ |
| | pBR322 ori, | | |
| | Amp^{R}) | | |
| scfv with | pET16b- | 15 | ScFvNdeF: 5′-TGATAACATATGCAGGTCCAACTGCAGC-3′ |
| 1x binding | ScFv1L | 17 | ScFv1LXhoR: 5′-CCGTTACTCGAGCCGCGCGGGGTGATCTAGGTC |
| loop | (PT7::scFv- | | CGCGCGGTCGTCGTCGTCATCATTAGTGGTGGTGGTGGTGGTGTT |
| (NdeI/XhoI) | (loop)₁; | | TGATCTCCAGCTTGGTCC-3′ |
| | pBR322 ori, | | |
| | Amp^{R}) | | |
| scfv with | pET16b- | 15 | ScFvNdeF: 5′-TGATAACATATGCAGGTCCAACTGCAGC-3′ |
| 3x binding | ScFv3L | 18 | 3L-1R: 5′-AGGTGAGCAACGGACATCCTTCACGGGTGATCTAGGTC |
| loop | (PT7::scFv- | | GTGAAGGCTCGATCGTCATCATTAGTGGTGGTGGTGGTGGTG-3′ |
| (NdeI/XhoI) | (loop)₃; | 19 | 3LXhoR: 5′-CCGTTACTCGAGTCGTAGAGCGGTGATCTAGGTGCTC |
| | pBR322 ori, | | TACGGACTGCGTTGCTCGGTGATCTAGGTGAGCAACGGACATCCT |
| | Amp^{R}) | | TCACG-3′ |
| BR2-ScFv | pET16b- | 20 | BR2ScFvNdeF: 5′-TGATAACATATGCGTGCTGGTCTGCAGT-3′ |
| (NdeI/XhoI) | BR2ScFv | 16 | ScFvXhoR: 5′-TCATTACTCGAGTCATCATTAGTGGTGGTGGTGGTG |
| | (PT7:: | | GTGTTTGATCTCCAGCTTGGTCC-3′ |
| | BR2scFv; | | |
| | pBR322 ori, | | |
| | Amp^{R}) | | |
| br2scfv with | pET16b- | 21 | BR2ScFvNdeF: 5′-TGATAACATATGCGTGCTGGTCTGCAGT |
| 1x binding | BRScFv1L | 17 | ScFv1LXhoR: 5′-CCGTTACTCGAGCCGCGCGGGGTGATCTAGGTC |
| loop | (PT7:: | | CGCGCGGTCGTCGTCGTCATCATTAGTGGTGGTGGTGGTGGTGTT |
| (NdeI/XhoI) | BR2scFv- | | TGATCTCCAGCTTGGTCC-3′ |
| | (loop)₁; | | |
| | pBR322 | | |
| | ori, Amp^{R}) | | |
| UGD | pET16b-UGD | 22 | UGDNdeF: 5′-TGATAACATATGAAAATCACCATTTCCGG-3′ |
| (NdeI/XhoI) | (PT7::ugd; | 23 | UGDXhoR: 5′-CCGTTACTCGAGTTATTAGTGGTGGTGGTGGTGGTG |
| | pBR322 ori, | | GTCGCTGCCAAAGAGATCG-3′ |
| | Amp^{R}) | | |
| ugd with | pET16b- | 22 | UGDNdeF: 5′-TGATAACATATGAAAATCACCATTTCCGG-3′ |
| 1x | UGD1L | 24 | UGD1LXhoR: 5′-CCGTTACTCGAGCCGCGCGGGGTGATCTAGGTC |
| binding loop | (PT7::ugd- | | CGCGCGGTCGTCGTCGTCATCATTAGTGGTGGTGGTGGTGGTGG |
| | loop)₁; | | TCGCTGCCAAAGAGATCG-3′ |
| | pBR322 ori, | | |
| | Amp^{R}) | | |
| Adh1p | pET16b- | 25 | AdhNdeF: 5′-TGATAACATATGTCTATCCCAGAAACTCAAAA-3′ |
| (NdeI/XhoI) | Adh | 26 | AdhXhoR: 5′-CCGTTACTCGAGTTATTAGTGGTGGTGGTGGTGGTG |
| | (PT7::adh1; | | TTTAGAAGTGTCAACAACGTATCGT-3′ |
| | pBR322 ori, | | |
| | Amp_{R}) | | |
| adh1p with | pET16b- | 25 | AdhNdeF: 5′-TGATAACATATGTCTATCCCAGAAACTCAAAA-3′ |
| 1x binding | Adh1L | 27 | Adx1LXhoR: 5′-CCGTTACTCGAGCCGCGCGGGGTGATCTAGGTCC |
| loop | (PT7::adh1- | | GCGCGGTCGTCGTCGTCATCATTAGTGGTGGTGGTGGTGGTGTTT |
| (NdeI/XhoI) | (loop)₁; | | AGAAGTGTCAACAACGTATCT-3′ |
| | pBR322 ori, | | |
| | Amp^{R}) | | |
| adh1p with | pET16b- | 25 | AdhNdeF: 5′-TGATAACATATGTCTATCCCAGAAACTCAAAA-3′ |
| 3x binding | Adh3L | 28 | 3L-1R: 5′-AGGTGAGCAACGGACATCCTTCACGGGTGATCTAGGTC |
| loop | (PT7::adh1- | | GTGAAGGCTCGATCGTCATCATTAGTGGTGGTGGTGGTGGTG-3′ |
| (NdeI/XhoI) | (loop)₃; | 29 | 3LXhoR: 5′-CCGTTACTCGAGTCGTAGAGCGGTGATCTAGGTGCTC |
| | pBR322 ori, | | TACGGACTGCGTTGCTCGGTGATCTAGGTGAGCAACGGACATCCT |
| | Amp^{R}) | | TCACG-3′ |
| UbiC | pET16b- | 30 | UbiCNdeF: 5′-TGATAACATATGCGATTGTTGCGTTTTTGTTGC-3′ |
| (NdeI/XhoI) | UbiC | 31 | UbiCXhoR: 5′-CCGTTACTCGAGTTATTAGTGGTGGTGGTGGTGGT |
| | (PT7::ubiC; | | GGTACAACGGTGACGCCGGTA-3′ |
| | pBR322 ori, | | |
| | Amp^{R}) | | |
| ubiC with | pET16b- | 30 | UbiCNdeF: 5′-TGATAACATATGCGATTGTTGCGTTTTTGTTGC-3′ |
| 1x binding | UbiC1L | 32 | UbiC1LXhoR: CCGTTACTCGAGCCGCGCGGGGTGATCTAGGTC |
| loop | (PT7::ubiC- | | CGCGCGGTCGTCGTCGTCATCATTAGTGGTGGTGGTGGTGGTGG |
| (NdeI/XhoI) | (loop)₁; | | TACAACGGTGACGCCGGTA-3′ |
| | pBR322 ori, | | |
| | Amp^{R}) | | |
| ubiC with | pET16b- | 30 | UbiCNdeF: 5′-TGATAACATATGCGATTGTTGCGTTTTTGTTGC-3′ |
| 3x binding | UbiC3L | 33 | 3L-1R: 5′-AGGTGAGCAACGGACATCCTTCACGGGTGATCTAGGT |
| loop | (PT7::ubiC- | | CGTGAAGGCTCGATCGTCATCATTAGTGGTGGTGGTGGTGGTG-3′ |
| (NdeI/XhoI) | (loop)₃; | 34 | 3LXhoR: 5′-CCGTTACTCGAGTCGTAGAGCGGTGATCTAGGTGCTC |
| | pBR322 ori, | | TACGGACTGCGTTGCTCGGTGATCTAGGTGAGCAACGGACATCCT |
| | Amp^{R}) | | TCACG-3′ |
| HIV1-Pr | pET16b- | 35 | HIVPrXbaF: 5′-ATTCTAAATCTAGATTATTCACTACGCGTTAAGGAG |
| (XbaI/XhoI) | HIVpr | | GTACGACATGCACCATCACCACCATCATCCTCAAATCACCCTGTG |
| | (PT7:: | | GC-3′ |
| | HIV1Pr, | 36 | HIVPrXhoR: 5′-CCGAATTACTCGAGTCATCATTAGAAGTTCAGGGT |
| | pBR322 ori, | | GCAACCGATCTGGGTCAGCATGTTACGACCGATGATGTTGATCGG |
| | Amp^{R}) | | GGTCG-3′ |
| hiv1pr with | pET16b- | 35 | HIVPrXbaF: 5′-ATTCTAAATCTAGATTATTCACTACGCGTTAAGGAG |
| 1x binding | HIVpr1L | | GTACGACATGCACCATCACCACCATCATCCTCAAATCACCCTGTG |
| loop | (PT7:: | | GC-3′ |
| (XbaI/XhoI) | HIV1Pr- | 37 | HIVPr1LXhoR: 5′-CCGTTACTCGAGCCGCGCGGGGTGATCTAGGTC |
| | (loop)₁; | | CGCGCGGTCGTCGTCGTCATCATTAGAAGTTCAGGGTGCAACCG-3′ |
| | pBR322 ori, | | |
| | Amp^{R}) | | |
| hiv1pr with | pET16b- | 35 | HIVPrXbaF: 5′-ATTCTAAATCTAGATTATTCACTACGCGTTAAGGAG |
| 3x binding | HIVpr3L | | GTACGACATGCACCATCACCACCATCATCCTCAAATCACCCTGTG |
| loop | (PT7:: | | GC-3′ |
| (NdeI/XhoI) | HIV1Pr- | 38 | HIVPrXho3LR: CCGAATTACTCGAGCCGCGCGGGGTGATCTAGG |
| | (loop)₃; | | TCCGCGCGGTCGTCGTCGTCATCATTAGAAGTTCAGGGTGCAACC-3′ |
| | pBR322 ori, | | |
| | Amp^{R}) | | |
| Leptin | pET16b-Lep | 39 | LepNdeF: 5′-TGATAACATATGCGATTGTTGCGTTTTTGTTGC-3′ |
| (NdeI/XhoI) | (PT7::LEP; | 40 | LepXhoR: 5′-CCGTTACTCGAGTTATTAGTGGTGGTGGTGGTGGTG |
| | pBR322 ori, | | GTACAACGGTGACGCCGGTA-3′ |
| | Amp^{R}) | | |
| leptin with | pET16b- | 39 | LepNdeF: 5′-TGATAACATATGCGATTGTTGCGTTTTTGTTGC-3′ |
| 1x binding | Lep1L | 41 | Lep1LXhoR: 5′-CCGTTACTCGAGCCGCGCGGGGTGATCTAGGTCC |
| loop | (PT7::LEP- | | GCGCGGTCGTCGTCGTCATCATTAGTGGTGGTGGTGGTGGTGGT |
| (NdeI/XhoI) | (loop)₁; | | ACAACGGTGACGCCGGTA-3′ |
| | pBR322 ori, | | |
| | Amp^{R}) | | |
| leptin with | pET16b- | 39 | LepNdeF: 5′-TGATAACATATGCGATTGTTGCGTTTTTGTTGC-3′ |
| 3x binding | Lep3L | 42 | 3L-1R: 5′-AGGTGAGCAACGGACATCCTTCACGGGTGATCTAGGTC |
| loop | (PT7::LEP- | | GTGAAGGCTCGATCGTCATCATTAGTGGTGGTGGTGGTGGTG-3′ |
| (NdeI/XhoI) | (loop)₃; | 43 | 3LXhoR: 5′-CCGTTABMPCTCGAGTCGTAGAGCGGTGATCTAGGTGCTC |
| | pBR322 ori, | | TACGGACTGCGTTGCTCGGTGATCTAGGTGAGCAACGGACATCCT |
| | Amp^{R}) | | TCACG-3′ |
| BMP2 | pET16b-BMP2 | 44 | BMP2NdeF: 5′-TGATAACATATGCAAGCCAAACACAAACAG-3′ |
| (NdeI/XhoI) | (PT7:: | 45 | BMP2XhoR: 5′-CCGTTACTCGAGTTATTAGTGGTGGTGGTGGTGGT |
| | BMP2; | | GGCGACACCCACAACCCTC-3′ |
| | pBR322 ori, | | |
| | Amp^{R}) | | |
| bmp2 with | pET16b- | 44 | BMP2NdeF: 5′-TGATAACATATGCAAGCCAAACACAAACAG-3′ |
| 1x binding | BMP21L | 46 | BMP21LXhoR: 5′-CCGTTACTCGAGCCGCGCGGGGTGATCTAGGTC |
| loop | (PT7:: | | CGCGCGGTCGTCGTCGTCATCATTAGTGGTGGTGGTGGTGGTGC |
| (NdeI/XhoI) | BMP2- | | GACACCCACAACCCTC-3′ |
| | (loop)₁; | | |
| | pBR322 ori, | | |
| | Amp^{R}) | | |
| bmp2 with | pET16b- | 44 | BMP2NdeF: 5′-TGATAACATATGCAAGCCAAACACAAACAG-3′ |
| 3x binding | BMP23L | 47 | 3L-1R: 5′-AGGTGAGCAACGGACATCCTTCACGGGTGATCTAGGTC |
| loop | (PT7:: | | GTGAAGGCTCGATCGTCATCATTAGTGGTGGTGGTGGTGGTG-3′ |
| (NdeI/XhoI) | BMP2- | 19 | 3LXhoR: 5′-CCGTTACTCGAGTCGTAGAGCGGTGATCTAGGTGCTC |
| | (loop)₃; | | TACGGACTGCGTTGCTCGGTGATCTAGGTGAGCAACGGACATCCT |
| | pBR322 ori, | | TCACG-3′ |
| | Amp^{R}) | | |
| sfGFP | pET16b- | 48 | sfGFPNdeF: 5′-TGATAACATATGCAAGCCAAACACAAACAG-3′ |
| (NdeI/XhoI) | sfGFP | 49 | sfGFPEcoR: 5′-AGGTCAGAATTCTCATCATTACGTAATACCTGCCG |
| | (PT7:: | | CATTC-3′ |
| | sfGFP; | | |
| | pBR322 ori, | | |
| | Amp^{R}) | | |
| C-sfGFP | pET16b- | 50 | CsfGFPXbaF: 5′-CCATGATCTAGAAATAATTTTGTTTAACTTTAAGA |
| (XbaI/NdeI) | CsfGFP | | AGGAGATATACCATGGTGCCCATCCAAAAAGTC-3′ |
| | (PT7:: | 51 | CsfGFPNdeR: 5′-CCGTTACATATGTCATCATTATGTAATCCCAGCA |
| | CsfGFP; | | GCATTTAC-3′ |
| | pBR322 ori, | | |
| | Amp^{R}) | | |
| N-sfGFP | pET16b- | 52 | sfGFPNdeF: 5′-TGATAACATATGCAAGCCAAACACAAACAG-3′ |
| | CsfGFP- | 53 | sfnGFPEcoR: 5′-AGGTCAGAATTCTCATCATTATTTTTCGTTCGGAT |
| | NsfGFP | | CTTTAGACA-3′ |
| | (PT7:: | | |
| | CsfGFP- | | |
| | NsfGFP; | | |
| | pBR322 ori, | | |
| | Amp^{R}) | | |
| nsgfp with | pET16b- | 48 | sfGFPNdeF: 5′-TGATAACATATGCAAGCCAAACACAAACAG-3′ |
| 3x binding | CsfGFP- | 54 | sfNGFP3L-1R: 5′-AGGTGAGCAACGGACATCCTTCACGGGTGATCT |
| loop | NsfGFP3L | | AGGTCGTGAAGGCTCGATCGTCATCATTATTTTTCGTTCGGATCTT |
| (NdeI/EcoRI) | (PT7:: | | TAGACA-3′ |
| | CsfGFP- | 55 | sfGFP3LEcoR: 5′-AGGTCAGAATTCTCGTAGAGCGGTGATCTAGG |
| | NsfGFP- | | TGCTCTACGGACTGCGTTGCTCGGTGATCTAGGTGAGCAACGGAC |
| | (loop)₃; | | ATCCTTCAC-3′ |
| | pBR322 ori, | | |
| | Amp^{R}) | | |

### Experimental Example 2. Construction of Expression Vector Encoding Fusion Protein of Chaperone and RNA-Binding Domain or Target Recombinant Protein for CRAS System

For a chaperone-recruiting mRNA scaffold (CRAS) system, an expression vector encoding a fusion protein of a chaperone and an RNA-binding domain or a target recombinant protein was constructed.

First, in order to prepare a medium-copy number vector, based on a pACYCDuet-1 vector having two strong T7 promoters (Novagen, Madison, Wis., USA), p15A which is a low-copy number origin of replication was replaced by pBR322 which is a medium-copy number origin of replication to prepare a pAMT7 vector.

Chaperone genes, dnaJ (SEQ ID NO: 74) and dnaK (SEQ ID NO: 75), were amplified by PCR, based on genomic DNA of E. coli BL21 (DE3). For high expression of dnaJ and dnaJ-KH, a synthetic ribosome-binding site was designed by using a ribosome-binding site (RBS) calculator, and incorporated into the 5′-terminus of DnaJNcoF primer.

To prepare the fusion protein of the chaperone and the RNA-binding domain, a gene (SEQ ID NO: 76) of Nova-1. KH3 RNA-binding domain (KH) having a sequence optimized for expression in E. coli was synthesized and bound downstream of dnaJ or dnaJK by recombinant PCR.

A gene encoding the target recombinant protein was amplified by PCR, based on various origins: scfv and br2-scfv were amplified based on pBR2ScFv (PloS one, 2013, 6, e66084), ugd and ubiC were amplified based on the genomic DNA of E. coli BL21(DE3), and adh1p was amplified based on the genomic DNA of S. cerevisae. HIV1-Pr gene was prepared by recombinant PCR (Protein expression and purification, 2015, 116, 59-65). tliA was amplified based on pTliA (Acs Catal, 2015, 5, 5016-5025). For green fluorescent protein (GFP) complementation assay, superfolder green fluorescent protein (sfGFP) (Bioneer, Daejeon, Korea) was used as a template to amplify the N-terminus and the C-terminus of sfGFP. For high expression of sfGFP, synthetic RBS and the N-terminus of sfGFP were designed by using an PBS calculator and incorporated into the 5′-terminus of sfGFPXbaP primer. The used primers are shown in Table 1.

For purification, 6×His-tag was added at the N-terminus of a forward primer for HIV1-Pr and at the C-terminus of a reverse primer for the target recombinant protein. For high expression of HIV1-Pr, synthetic RBS was designed by using the RBS calculator, and included at the 5′-terminus of HIVPrXbaF primer. PCR products and expression vectors corresponding thereto were digested with restriction enzymes in Table 1. Then, the chaperone was ligated to pAMT7 and the recombinant protein was ligated to pET16b.

### Experimental Example 3. Preparation of RNA Scaffold for CRAS System

For the chaperone-recruiting mRNA scaffold (CRAS) system, an RNA scaffold was prepared. In detail, a sequence of a binding loop for KH domain used in the RNA scaffold system was designed by RNA Designer and mFold (Journal of molecular biology, 2004, 336, 607-624; Science 1989, 244, 48-52). A sequence constraint is as follows;

5′-NNNNNNNNACCTAGATCACC(SEQ ID NO: 77)NNNNNNNN-3′, wherein N represents an arbitrary nucleotide of 8 bp for a stem structure, and a sequence underlined represents a loop structure including a binding sequence for the KH RNA-binding domain.

In the case of an RNA scaffold system having a plurality of binding loops, a stem-loop structure was designed by a similar approach to add a 5-nt spacer between respective stem-loop structures. A stem-loop structure for the target protein gene was prepared by PCR using a reverse primer having a sequence of the stem-loop structure in 3′-UTR.

### Experimental Example 4. Preparation of CLEX System

CLEX system of DnaJ chaperone and recombinant protein (ScFv, UbiC, Leptin, HIV1-Pr, BMP2, or TliA) was prepared in two different arrangements in which DnaJ was placed in the first or second cistron. A 12-nt region (5′-GAGGAGGTGGAA-3′, SEQ ID NO: 79) encoding an amino acid sequence of EEVE (EEVE, SEQ ID NO: 78) and including a Shine-Dalgarno (SD) sequence (underlined) was introduced at the C-terminus of the gene in the first cistron in order to improve translation initiation of the gene in the second cistron. To secure translational coupling and to promote interaction between DnaJ and the recombinant protein, the stop codon of the first cistron and the start codon of the second cistron were overlapped through 1 nt (5′-TAATG-3′). To prepare the CLEX system, the DnaJ chaperone and the recombinant protein were assembled by recombinant PCR. Primer sequences are shown in Table 2. Each PCR product and pET16b were digested by restriction enzyme described in Table 2, and ligated to the expression vector.

TABLE 2

| | | SEQ | |
|---|---|---|---|
| | | ID | |
| Proteins | Plasmid | NO | Primers (5′-3′) |
| DnaJ/ScFv | pET16b- | 56 | DnaJNdeF: 5′-TGATAACATATGGCTAAGCAAGATTATTACG-3′ |
| | DnaJScFv | 57 | ScFvFpol: 5′-GTTTTTTGACGACCTGACCCGCGAGGAGGTGG |
| | (pET::dnaJ- | | AATAATGCAGGTCCAACTGCAGC-3′ |
| | ScFv; pBR322 | 16 | ScFvXhoR: 5′-TCATTACTCGAGTCATCATTAGTGGTGGTGGT |
| | ori, Amp^{R}) | | GGTGGTGTTTGATCTCCAGCTTGGTCC-3′ |
| ScFv/DnaJ | pET16b- | 15 | ScFvNdeF: 5′-TGATAACATATGCAGGTCCAACTGCAGC-3′ |
| (NdeI/XhoI) | ScFvDnaJ | 58 | DnaJFpol: 5′-CCACCACCACCACCACCACGAGGAGGTGGAATA |
| | (PT7::ScFv- | | ATGGCTAAGCAAGATTATTACG-3′ |
| | dnaJ; pBR322 | 2 | DnaJXhoR: |
| | ori, Amp^{R}) | | 5′-ATTTCACTCGAGTTATTAGCGGGTCAGGTCGTCAAA-3′ |
| DnaJ/UbiC | pET16b- | 56 | DnaJNdeF: 5′-TGATAACATATGGCTAAGCAAGATTATTACG-3′ |
| (NdeI/XhoI) | DnaJUbiC | 59 | UbiCFpol: 5′-GTTTTTTGACGACCTGACCCGCGAGGAGGTGG |
| | (PT7::dnaJ- | | AATAATGCGATTGTTCGCGTTTTTGTTGC-3′ |
| | UbiC; pBR322 | 31 | UbiCXhoR: 5′-CCGTTACTCGAGTTATTAGTGGTGGTGGTGGT |
| | ori, Amp^{R}) | | GGTGGTACAACGGTGACGCCGGTA-3′ |
| UbiC/DnaJ | pET16b- | 30 | UbiCNdeF: |
| (NdeI/XhoI) | UbiCDnaJ | | 5′-TGATAACATATGCGATTGTTGCGTTTTTGTTGC-3′ |
| | (PT7::UbiC- | 58 | DnaJFpol: 5′-CACCACCACCACCACCACGAGGAGGTGGAATA |
| | dnaJ; pBR322 | | ATGGCTAAGCAAGATTATTACG-3′ |
| | ori, Amp^{R}) | 2 | DnaJXhoR: |
| | | | 5′-ATTTCACTCGAGTTATTAGCGGGTCAGGTCGTCAAA-3′ |
| DnaJ/HIV1- | pET16b- | 56 | DnaJNdeF: 5′-TGATAACATATGGCTAAGCAAGATTATTACG-3′ |
| Pr | DnaJHIV1Pr | 60 | HIVPrFpol: |
| (NdeI/XhoI) | PT7::dnaJ- | | 5′-GTTTTTTGACGACCTGACCCGCGAGGAGGTGGAATAATG |
| | HIV1Pr, | | CACCATCACCACCATCATCCTCAAATCACCCGTGGC-3′ |
| | pBR322 ori, | 36 | HIVPrXhoR: 5′-CCGAATTACTCGAGTCATCATTAGAAGTTCAG |
| | Amp^{R}) | | GGTGCAACCGATCTGGGTCAGCATGTTACGACCGATGATG |
| | | | TTGATCGGGGTCG-3′ |
| HIV1-Pr/ | pET16b- | 35 | HIVPrXbaF: 5′-ATTCTAAATCTAGATTATTCACTACGCGTTAAG |
| DnaJ | HIV1PrDnaJ | | GAGGTACGACATGCACCATCACCACCATCATCCTCAAATCA |
| (XbaI/XhoI) | (PT7::HIV1Pr- | | CCCTGTGGC-3′ |
| | dnaJ; pBR322 | 61 | DnaJFpolHIVPr: 5′-CGGTTGCACCCTGAACTTCGAGGAGGTG |
| | ori, Amp^{R}) | | GAATAATGGCTAAGCAAGATTATTACG-3′ |
| | | 2 | DnaJXhoR: 5′-ATTTCACTCGAGTTATTAGCGGGTCAGGTCGT |
| | | | CAAA-3′ |
| DnaJ/Leptin | pET16b- | 56 | DnaJNdeF: 5′-TGATAACATATGGCTAAGCAAGATTATTACG-3′ |
| (NdeI/XhoI) | DnaJLep | 62 | LepFpol: |
| | (PT7::dnaJ- | | 5′-GTTTTTTGACGACCTGACCCGCGAGGAGGTGGAATAATG-3′ |
| | LEP; pBR322 | | CGATTGTTGCGTTTTTGTTGC-3′ |
| | ori, Amp^{R}) | 40 | LepXhoR: 5′-CCGTTACTCGAGTTATTAGTGGTGGTGGTGGT |
| | | | GGTGGTACAACGGTGACGCCGGTA-3′ |
| Leptin/DnaJ | pET16b- | 39 | LepNdeF: |
| (NdeI/XhoI) | LepDnaJ (PT7:: | | 5′-TGATAACATATGCGATTGTTGCGTTTTTGTTGC-3′ |
| | LEP-dnaJ; | 58 | DnaJFpol: 5′-CACCACCACCACCACCACGAGGAGGTGGAATA |
| | pBR322 ori, | | ATGGCTAAGCAAGATTATTACG-3′ |
| | Amp^{R}) | 2 | DnaJXhoR: |
| | | | 5′-ATTTCACTCGAGTTATTAGCGGGTCAGGTCGTCAAA-3′ |
| DnaJ/BMP2 | pET16b- | 56 | DnaJNdeF: 5′-TGATAACATATGGCTAAGCAAGATTATTACG-3′ |
| (NdeI/XhoI) | DnaJBMP2 | 63 | BMP2FPol: 5′-GTTTTTTHACGACCTGACCCGCGAGGAGGTG |
| | (PT7::dnaJ- | | GAATAATGCAAGCCAAACACAAACAG-3′ |
| | BMP2; pBR322 | 45 | BMP2XhoR: 5′-CCGTTACTCGAGTTATTAGTGGTGGTGGTGG |
| | ori, Amp^{R}) | | TGGTGGCGACACCCACAACCCTC-3′ |
| BMP2/DnaJ | pET16b- | 44 | BMP2NdeF: 5′-TGATAACATATGCAAGCCAAACACAAACAG-3′ |
| (NdeI/XhoI) | BMP2DnaJ | 58 | DnaJFpol: 5′-CACCACCACCACCACCACGAGGAGGTGGAATA |
| | (PT7::BMP2- | | ATGGCTAAGCAAGATTATTACG-3′ |
| | dnaJ; pBR322 | 2 | DnaJXhoR: |
| | ori, Amp^{R}) | | 5′-ATTTCACTCGAGTTATTAGCGGGTCAGGTCGTCAAA-3′ |
| DnaJ/Adh1p | pET16b- | 56 | DnaJNdeF: |
| (NdeI/XhoI) | DnaJAdh | | 5′-TGATAACATATGGCTAAGCAAGATTATTACG-3′ |
| | (PT7::dnaJ- | 64 | AdhFPol: 5′-GTTTTTTGACGACCTGACCCGCGAGGAGGTGG |
| | adh1; pBR322 | | AATAATGTCTATCCCAGAAACTCAAAA-3′ |
| | ori, Amp^{R}) | 26 | AdhXhoR: 5′-CCGTTACTCGAGTTATTAGTGGTGGTGGTGGT |
| | | | GGTGTTTAGAAGTGTCAACAACGTATCT-3′ |
| Adh1p/DnaJ | pET16b- | 25 | AdhNdeF: |
| (NdeI/XhoI) | AdhDnaJ | | 5′-TGATAACATATGTCTATCCCAGAAACTCAAAA-3′ |
| | (PT7::adh1- | 58 | DnaJFpol: 5′-CACCACCACCACCACCACGAGGAGGTGGAATA |
| | dnaJ; pBR322 | | ATGGCTAAGCAAGATTATTACG-3′ |
| | ori, Amp^{R}) | 2 | DnaJXhoR: |
| | | | 5′-ATTTCACTCGAGTTATTAGCGGGTCAGGTCGTCAAA-3′ |
| TliA | pET16b-TliA | 65 | TliANdeF: 5′-TGATAACATATGCATCATCATCATCTCTCATC |
| (NdeI/EcoRI) | (PT7::tliA; | | ATCACAGCA-3′ |
| | pBR322 ori, | 66 | TliAEcoR: 5′-CTGAGAATTCTCATCATTAACTGATCAGCACAC |
| | Amp^{R}) | | CCTCGCTCC-3′ |
| DnaJ/TliA | pET16b- | 56 | DnaJNdeF: |
| (NdeI/EcoRI) | DnaJTliA | | 5′-TGATAACATATGGCTAAGCAAGATTATTACG-3′ |
| | (PT7::dnaJ- | 67 | TliAFPol: 5′-GTTTTTTGACGACCTGACCCGCGAGGAGGTGG |
| | tliA, pBR322 | | AATAATG CATCATCATCATCATCATCATCATCACAGCA-3′ |
| | ori, Amp^{R}) | 66 | TliAEcoR: 5′-CTGAGAATTCTCATCATTAACTGATCAGCACAC |
| | | | CCTCGCTCC-3′ |
| TliA/DnaJ | pET16b- | 65 | TliANdeF: 5′-TGATAACATATGCATCATCATCATCTCTCATC |
| (NdeI/EcoRI) | TliADnaJ | | ATCACAGCA-3′ |
| | (PT7::tilA- | 68 | DnaJTliAFpol: 5′-GGAGCGAGGGTGTGCTGATCAGTGAGGA |
| | dnaJ, pBR322 | | GGTGGAATAATGGCTAAGCAAGATTATTACG-3′ |
| | ori, Amp^{R}) | 69 | DnaJEcoR: |
| | | | 5′-CTGAGAATTCTTATTAGCGGGTCAGGTCGTCAA A-3′ |
| TliA | pET16b-TliA1 | 65 | TliANdeF: 5′-TGATAACATATGCATCATCATCATCTCTCATC |
| (NdeI/EcoRI) | (PT7::tliA | | ATCACAGCA-3′ |
| | (aa 1-300); | 70 | TliA1EcoR: 5′-CCGTTAGAATTCTCATCATTAGTCGGTGGTCG |
| | pBR322 ori, | | ACTCGTG-3′ |
| | Amp^{R}) | | |
| DnaJ/TliA1 | pET16b- | 56 | DnaJNdeF: 5′-TGATAACATATGGCTAAGCAAGATTATTACG-3′ |
| (NdeI/EcoRI) | DnaJTliA1 | 71 | TilA1FPol: 5′-AGTTTTTTGACGACCTGACCCGCACCGGAGGT |
| | (PT7::dnaJ- | | ACATAATGCATCATCATCATCATCATCATCAATCACAGCA-3′ |
| | tilA (aa 1- | 72 | TilA1EcoR: 5′-CCGTTAGAATTCTCATCATTAGTCGGTGGTCG |
| | 300); pBR322 | | ACTCGTG-3′ |
| | ori, Amp^{R}) | | |
| TliA2 | pET16b-TliA2 | 72 | TliA2NdeF: 5′-TGATAACATATGAACATCGTCAGCTTCAACG-3′ |
| (NdeI/EcoRI) | (PT7:: tilA | 66 | TliAEcoR: 5′-CTGAGAATTCTCATCATTAACTGATCAGCACAC |
| | (aa 301-493); | | CCTCGCTCC-3′ |
| | pBR322 ori, | | |
| | Amp^{R}) | | |

### Experimental Example 5. Method of Measuring Protein Solubilization

E. coli BL2(DE3) was co-transformed with the plasmid of the chaperone and the plasmid of the target recombinant protein. Thereafter, the E. coli was inoculated in 3 ml of LB (Lysogenic Broth) medium (10 g/l tryptone, 5 g/l yeast; extract, and 10 g/l sodium chloride) supplemented with ampicillin and chloramphenicol at a final concentration of 50 μg/ml and 25 μg/ml, respectively and cultured in a rotary shaker (200 rpm) at 37° C. overnight. Then, 1 ml of the culture was inoculated in 100 ml of LB medium supplemented with ampicillin and chloramphenicol at a final concentration of 50 μg/ml and 25 μg/ml, respectively and then cultured at 200 rpm and 37° C. When OD₆₀₀ reached 0.5-0.6, 0.5 mM isopropyl β-d-1-thiogalactopyranoside (IPTG) was added to induce expression of the chaperone and the target protein, and then, further cultured for 4 hours (37° C., 200 rpm). Thereafter, when OD₆₀₀ value reached 2, 3 ml of cell culture (including about 1.6×10⁹ cells) was centrifuged and resuspended in 500 μl of 10 mM Tris-EDTA (TE) buffer (pH 7.6), followed by sonication. Soluble and insoluble fractions were separated by centrifugation (21, 600×g, 15 min, 4° C.). An insoluble pellet was washed with 1%. Triton X-100 twice, and resuspended in 10 mM TE buffer (pH 7.6) and used as an insoluble fraction. Solubility of the target recombinant protein was measured by dodecyl sodium sulfate polyacrylamide gel electrophoresis (SDS-PAGE). Band intensity of the whole cell lysates and the soluble fractions of the target recombinant proteins stained with Coomassie Blue in the SDS-PAGE gel were compared by using an ImageJ software to calculate relative percentages of the soluble target recombinant proteins.

### Experimental Example 6. Method of Purifying Protein

E. coli BL21(DE3) having pAMT7-DnaJ-KH was cultured in an LB medium supplemented with chloramphenicol at a final concentration of 25 μg/ml. The E. coli was cultured in a rotary shaker (200 rpm) at 37° C. overnight. Then, 1 ml of the culture was inoculated in 100 ml of LB medium supplemented with chloramphenicol at a final concentration of 25 μg/ml, and cultured at 200 rpm at 37° C. When OD₆₀₀ reached 0.6, 0.5 mM IPTG was added to induce expression of the DnaJ-KH protein, and then, further cultured for 4 hours (37° C., 200 rpm). Thereafter, 20 ml of the cells were harvested by centrifugation, and resuspended in 1× Native IMAC lysis buffer (Biorad, Hercules, Calif., USA), followed by sonication. Transparent cell lysate was further centrifuged (21,600×g, 15 min, 4° C.). DnaJ-KH was purified from a soluble fraction by a Native IMAC method of an automated Profinia™ protein purification system (Biorad, Hercules, Calif., USA). Thereafter, the purified DnaJ-KH was dialyzed against phosphate-buffered saline (PBS) at pH 7.4 and 25 mM Tris-HCl buffer (pH 8.8) containing 5 mM DTT, 100 mM NaCl, and 10% glycerol.

### Experimental Example 7. GFP Complementation Assay

Correct folding of the recombinant protein in the CRAS system was evaluated by a known GFP complementation assay (Nature methods, 2012, 9, 671-675) with slight modification. E. coli BL21(DE3) having pAMT7-DnaJK-KH and pET16b-sfGFP/pET16b-CsfGFP-NsfGFP/pET16b-CsfGFP-NsfGFP3L was inoculated in an LB medium supplemented with ampicillin and chloramphenicol at a final concentration of 50 μg/ml and 25 μg/ml, respectively and cultured in a rotary shaker (200 rpm) at 37° C. overnight. Then, 1 ml of the culture was inoculated in 100 ml of LB medium supplemented with ampicillin and chloramphenicol at a final concentration of 50 μg/ml and 25 μg/ml, respectively, and cultured at 200 rpm at 37° C. When OD₆₀₀ reached 0.6, 0.5 mM IPTG was added to induce expression of DnaJ-KH and the target protein, and then, further cultured for 4 hours (37° C., 200 rpm). Thereafter, 1 ml of the cells were harvested by centrifugation, and resuspended in 500 μl of PBS (pH 7.4) and diluted until OD₆₀₀ reached 1. Thereafter, the cells were loaded in a 96-well black plate (SPL Life Sciences, Pocheon, Korea). Tecan Infinite F200 PRO instrument (Tecan Group Ltd., Mannedorf, Switzerland) was used to measure fluorescence intensity of each well (λ_{exc}=488 nm/λ_{exc}=530 nm).

### Experimental Example 8. Electric Mobility Shift Assay (EMSA)

mRNA of anti p21ras-ScFvd having no loop, one loop, or three loops was prepared by using a HiScribe™ T7 High Yield RNA Synthesis Kit (New England Biolab, Beverly, Mass.). PBS (pH 7.4) having 0.1 nM mRNA and 200 μM of purified DnaJ-KH were mixed. Thereafter, the mixture was incubated at 25° C. for 30 minutes, and analyzed by electrophoresis on 2% agarose gel in Tris-boric acid-EDTA buffer. Electrophoresis was performed at constant 50 V for 20 minutes. Thereafter, the gel was visualized by using a Gel-Doc gel documentation system (Bio-Rad, Hercules, Calif.).

### Experimental Example 9. Method of Measuring Leptin Activity

E. coli BL21(DE3) having pET16b-DnaJLep or pET16b-Lep was cultured in an LB medium supplemented with ampicillin at a final concentration of 50 μg/ml in a rotary shaker (200 rpm) at 37° C. overnight. Then, 1 ml of the culture was inoculated in 100 ml of LB medium supplemented with ampicillin at a final concentration of 50 μg/ml, and cultured at 200 rpm at 37° C. When OD₆₀₀ reached 0.6, 0.5 mM IPTG was added and then, further cultured for 4 hours (37° C., 200 rpm) to induce expression of DnaJ and leptin. Thereafter, 20 ml of the cells were harvested by centrifugation, and resuspended in 1×PBS (pH 7.4), followed by sonication. Transparent cell lysate was further centrifuged (21, 600×g, 15 min, 4° C.). Activity of the purified leptin was analyzed by ELISA. 40 μg of a soluble fraction of the transparent cell lysate (8 mg/ml) was loaded in a 96-well plate (Thermo Fisher Scientific, Waltham, Mass., USA) with carbonate-bicarbonate buffer at 37° C. for 1 hour. Thereafter, the plate was washed with 1×PBS-T (1×PBS containing 0.5% Tween-20) three times, and then each well was blocked with bovine serum albumin (Invitrogen, Carlsbad, Calif., USA) for 1 hour. Thereafter, an anti-leptin antibody (Abeam, Cambridge, UK) was added to each well, and incubated at room temperature for 1 hour. After washing the plate, the plate was incubated with a horseradish peroxide-conjugated anti-rabbit monoclonal antibody (Cell Signaling Technology, Beverly, Mass., USA) at room temperature for 1 hour. Thereafter, the plate was washed with 1×PBS-T four times, and to initiate peroxidase reaction, 100 μl of TMB (3,3′,5,5′-Tetramethylbenzidine) peroxidase substrate (BD, Franklin Lakes, N.J., USA) was added. To stop the reaction, 50 μl of 2 M H₂SO₄ was added to each well. An ELISA reader (Infinite M200 PRO; Tecan Group Ltd., Mannedorf, Switzerland) was used to measure absorbance of each well at 450 nm wavelength.

### Example 1. Result of Applying Various Insoluble Proteins to CRAS System

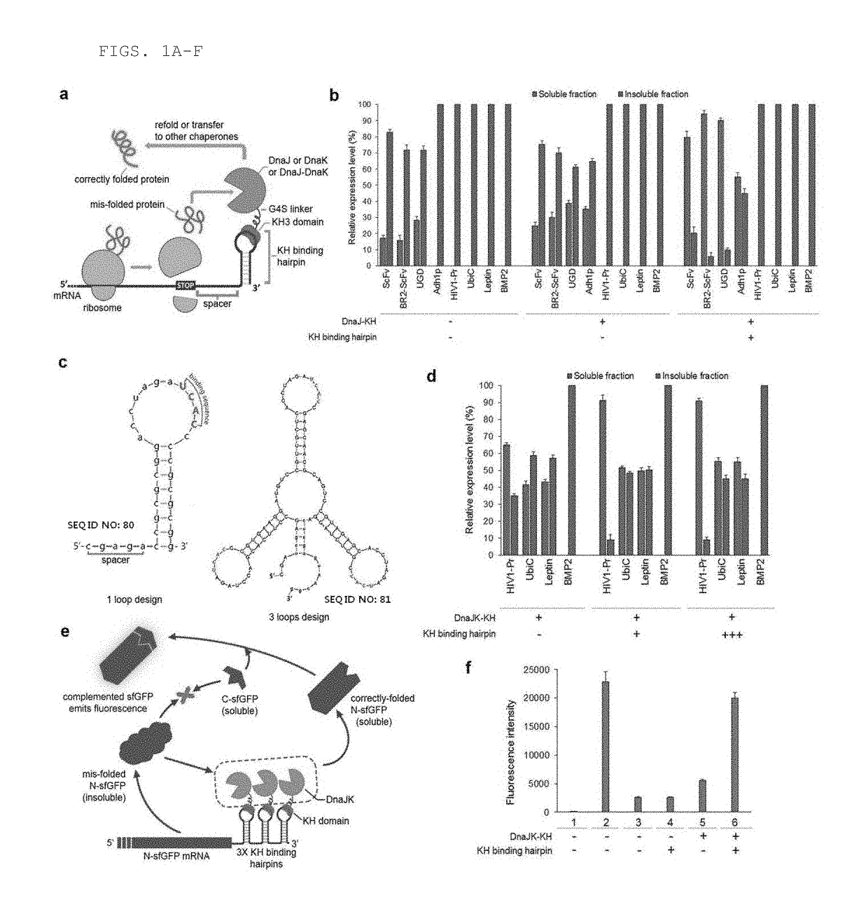

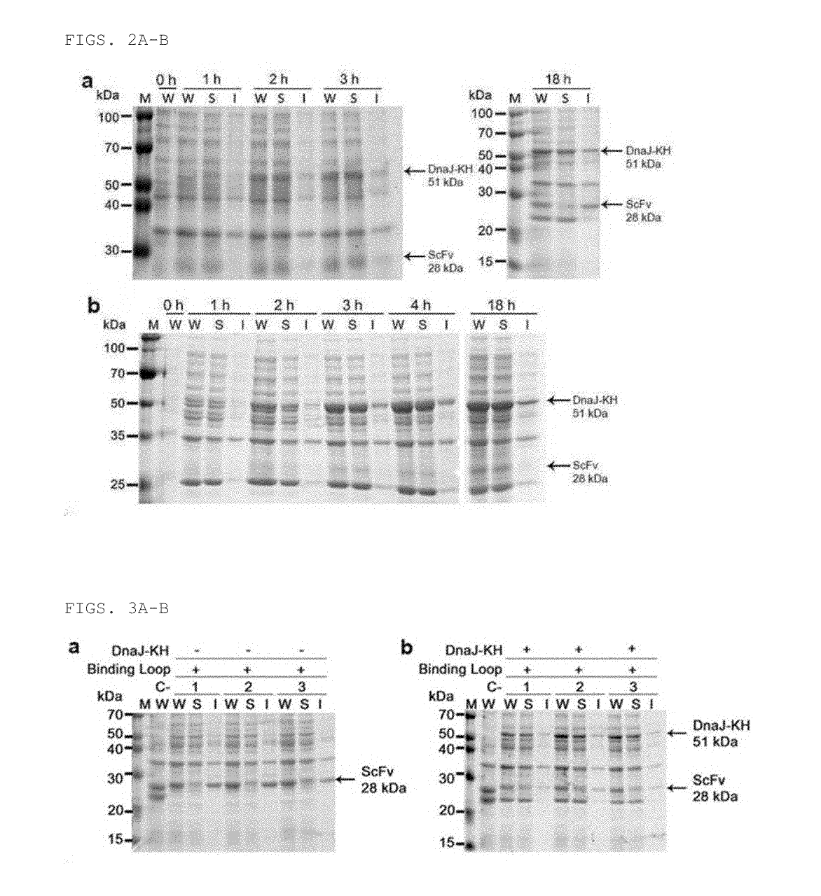

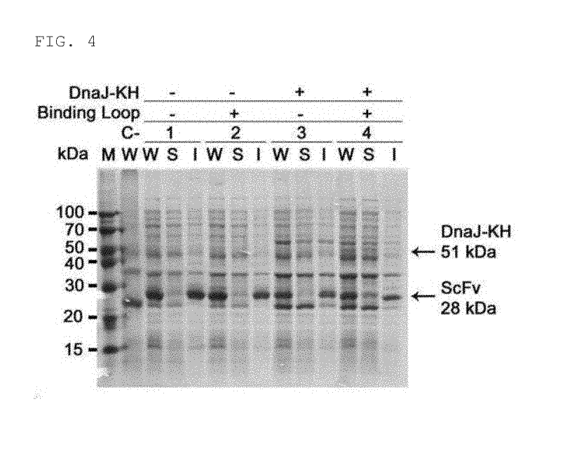

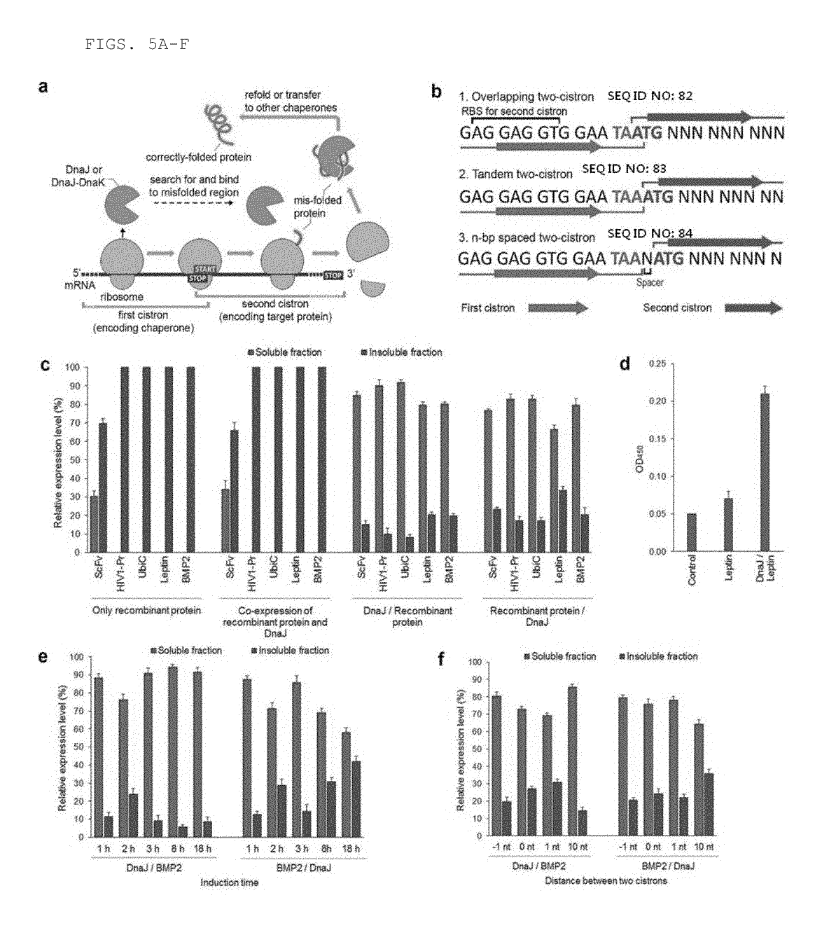

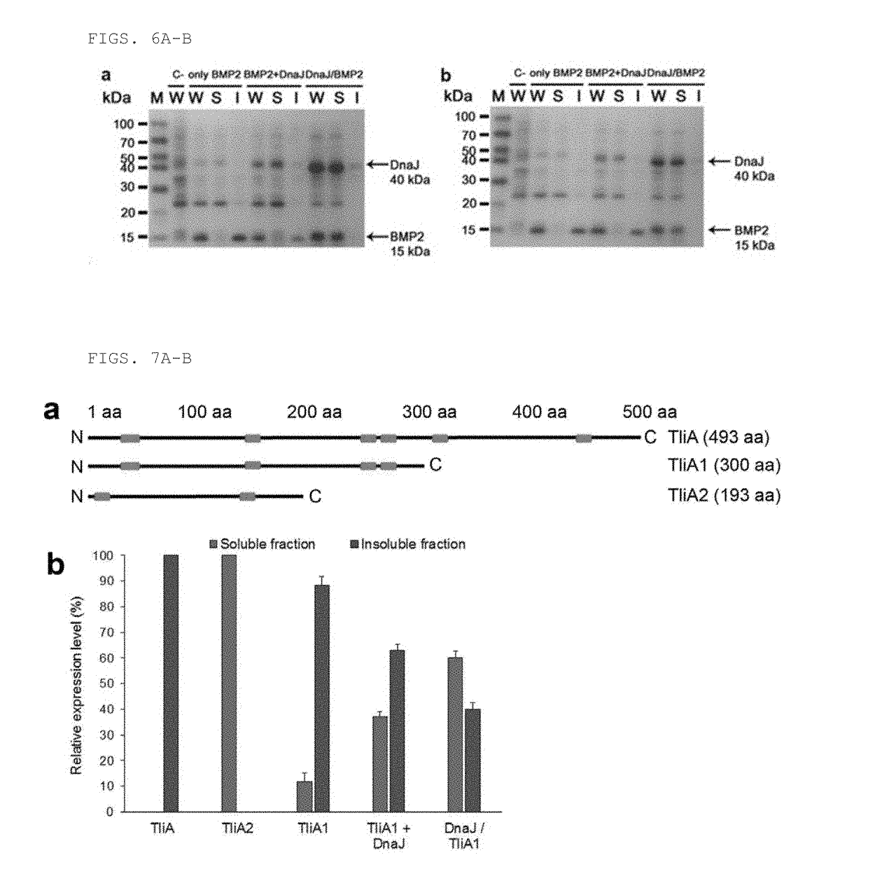

DnaJ, which is an essential element for a molecular chaperone system in bacteria, interacts with a translated or misfolded protein and promotes interaction between foldase chaperone such as DnaK or GroESL and a protein in order to produce a correctly folded protein. For rapid folding immediately after translation, DnaJ was placed at the translation termination site of the target protein to increase spatial proximity between DnaJ and the target protein. For anchoring, human Nova-1 protein-derived sequence-specific RNA-binding domain (KH) was bound with DnaJ (FIG. 1A). Interaction between DnaJ-KH (complex of DnaJ and KH) and 3′UTR hairpin loop was examined by a gel retardation assay. In order to increase solubility of insoluble recombinant proteins such as anti-Ras ScFv (single chain variable fragment), a complex of ScFv and an antimicrobial peptide BR2 (BR2-ScFv), UDP-6-glucose dehydrogenase (USD), UbiC (pyruvate chorismate lyase), BMP2 (bone morphogenetic protein 2), leptin, Adh1p (alcohol dehydrogenase 1p), HIV-1 protease (HIV1-Pr) of E. coli, etc., these proteins were applied to the CRAS (chaperone recruiting mRNA scaffold) system.

As a result, when the CRAS system was applied, solubility of ScFv, BR2-ScFv, and UGD was greatly increased, as compared with co-expression of the protein and DnaJ-KH without spatial restriction (without binding loops) or single expression of the protein (FIG. 1B).

### Example 2. Result of Solubilization According to Number of 3′UTR Hairpin Loop and Distance Between Stop Codon and First Loop in CRAS System

The CRAS system did not increase solubility of Adh1p, HIV1-Pr, UbiC, Leptin, and BMP2 (FIG. 1B). To solve this problem, the number of 3′UTR hairpin loop was increased. First, a number of 3′UTR hairpin loops were designed and verified through computer simulation to create desired structures (FIG. 1C).

Up to three of 3′UTR hairpin loops showed no significant effect on solubility increase of the protein. However, in this case, a percentage of the soluble fraction of ScFv was maintained constant for 18 hours of expression, whereas the solubility of the protein having one 3′UTR hairpin loop was gradually reduced (FIGS. 2A-B), which is very important in increasing the protein production yield.

Further, a space of 30 nt or less between the stop codon and the first loop structure had no influence on solubility increase of ScFv (FIGS. 3A-B), indicating that the system has flexibility about the distance between the stop codon and DnaJ.

### Example 3. Result of Applying CRAS System in ΔdnaJ Strain and ΔdnaK Strain

DnaK chaperone system is naturally present in E. coli, and the system of the present invention anchors the DnaJ chaperone to a translation site, and therefore, dnaJ or dnaK was deleted in the genome of E. coli, and then whether ScFv solubility was increased or not was analyzed by using CRAS system. Since only DnaJ targeting mRNA is able to efficiently interact with ScFv, and the function of the CRAS system may be reduced in ΔdnaK strain due to lack, of foldase chaperone, it was predicted that the same solubility enhancement efficiency would be observed in the ΔdnaJ strain. The ΔdnaJ strain was as predicted, but the ΔdnaK strain showed slightly increased solubility of ScFV (FIG. 4), suggesting that the CRAS system increased foldase activity of DnaJ so that DnaJ alone functions as the chaperone to increase solubility of the recombinant protein.

### Example 4. Result of Applying DnaJ-DnaK-KH Complex to CRAS System

It was intended to increase efficiency of the CRAS system by a method of restricting the spatial extent of the chaperone proteins and by a method of increasing the turnover number of each chaperone. To this end, a chimeric chaperone DnaJ-DnaK-KH (DnaJK-KH), which is a complex of DnaJ, DnaK, and KH, was prepared and applied to the CRAS system.

As a result, it was found that solubility of the insoluble proteins was very greatly increased, and up to 90% of the expressed proteins had soluble forms (FIG. 1D). Further, as the numbers of DnaJK-KH and KH hairpin loops were increased, solubility of the target proteins was rapidly increased after protein expression, as compared with DnaJ-KH (FIG. 1D), suggesting that the number of chaperones adjacent to the translation process is a limiting factor in the folding reactions.

### Example 5. Result of GFP Complementation Assay in CRAS System

To examine functional folding of the target protein in vivo, a GFP complementation assay was designed by splitting superfolder green fluorescent protein (sfGFP) into N-terminus (N-sfGFP) and C-terminus (C-sfGFP) (FIG. 1E). Since N-sfGFP has low solubility, it was selected as a target protein to investigate function of the CRAS system. A fluorescence level represents a level, of solubilized functional N-sfGFP. When DnaJK-KH and N-sfGFP interacting therewith are coexpressed with C-sfGFP, fluorescence intensity of cells coexpressing the two fragments was increased twice. In contrast, when three loops were introduced into 3′UTR of N-sfGFP interacting with DnaJ-KH, fluorescence intensity was increased about 8 times, which corresponds to 90% of the fluorescence intensity emitted from the full length protein of sfGFP, indicating that the CRAS system may increase not only solubility but also expression of functional active proteins (FIG. 1F).

### Example 6

### Example 6-1. Result of Applying CLEX System

To demonstrate importance of spatial restriction to the chaperone and the substrate without limiting the number of chaperone adjacent to the translational machinery, a CLEX (chaperone-substrate co-localized expression) system which is a translationaily coupled two-cistron expression system capable of solubilizing very insoluble proteins was prepared (FIG. 5A). The overlapping (5′-TAATG-3′) of the stop codon and the start codon of the first and second cistrons prevents a transcript from exiting the ribosomal complex and allows translation of the second cistron. Further, the CLEX system was designed such that DnaJ was linked to the target protein-encoding gene of the first or second cistron, and the first cistron has 5′-GAGGAGGTGGAA-3′ sequence (SEQ ID NO: 79) (underlined sequence: RBS or Shine-Dalgarno sequence) including the ribosome-binding site (RBS) for the second cistron. With regard to an integration sequence between two cistrons, two overlapping cistrons, two cistrons in tandem, and two cistrons having a space of n bp therebetween were designed (FIG. 5B).

As a result, the CLEX system having the overlapping stop-start codons was very effective in solubilizing very insoluble proteins including BMP2, which are not solubilized by the CRAS system. On average, 60% to 90% of the recombinant proteins were obtained as soluble forms (FIG. 5C). Particularly, correct folding of leptin was confirmed by using a structure-specific antibody according to the method of Experimental Example 9 (FIG. 5D).

Further, the role of DnaJ was determined by the order of the two cistrons, which generates a difference in the expression level and reaction time (FIG. 5C). When the chaperone was expressed from the first cistron, a larger number of soluble fractions were identified. This is because the available chaperone may be prepared prior to translation of the second cistron. Further, the arrangement of DnaJ-BMP2 showed longer protein expression (up to 18 hours after induction) than its reverse arrangement (FIG. 5E), indicating that the timely interaction between the chaperone and the protein is more important than the amount of constituents involved in the chaperone reaction.

Further, it was confirmed that the protein solubility may be increased by increasing a sequence between the genes to 10 nt (FIG. 5F). The nucleotides added between the two genes changed not only a stoichiometric ratio between the two proteins but also spatial proximity between DnaJ and the target recombinant protein, thereby changing the secondary structure of the transcript and affinity for translation initiation of the second cistron.

### Example 6-2. Result of Applying CLEX System in ΔdnaK Strain

In order to identify the role of DnaK in solubilization of the recombinant protein by the CLEX system, ΔdnaK strain was used (FIGS. 6A-B).

As a result, it was confirmed that DnaJ was able to sufficiently solubilize BMP2 protein even in the absence of DnaK in the CLEX system, indicating that DnaJ has foldase activity independently of DnaK or other chaperone system is involved in the protein folding in E. coli. Accordingly, it can be seen that the CLEX system using the single chaperone DnaJ has very high efficiency.

### Example 7. Result of Applying TliA 1 to CLEX System

Although various recombinant proteins were successfully solubilized, solubility of a high molecular weight protein such as TliA (52 kDa lipase) was not highly increased. This is because high molecular weight proteins tend to have many domains that are likely to be misfolded, and the system of the present invention is insufficient to promote the interaction between the chaperone and a number of substrates before irreversible misfolding. To examine this, tliA gene was cut into two fragments (tliA1, tliA2), and then applied to the system of the present invention.

As a result, TliA1 was very insoluble whereas TliA2 was soluble (FIGS. 3A and 3B). In the CLEX system where tilA1 was expressed from the second citron, about 60% of TliA1 was solubilized. In contrast, when TliA1 and DnaJ were simply coexpressed, 25% of TliA1 was solubilized (FIG. 3B), suggesting that the intrinsic properties of TliA, such as protein length and domain arrangement, are the main causes of insolubility, and the solubility of high molecular weight proteins may be increased through the system of the present invention which is capable of sufficiently exposing an area which is likely to be misfolded to the chaperone.

The CRAS system places the chaperone at the 3′UTR of mRNA whereas the CLEX system produces a new chaperone molecule together with the target protein to promote a rapid interaction therebetween. Considering solubilization activity, the CLEX system is superior to the CRAS system. However, the CLEX system is limited in solubilizing a number of proteins at the same time, because dnaJ must be cloned into two cistron systems for each recombinant protein. In contrast, the CRAS system having three loops and DnaJK-KH may improve the solubility of many proteins expressed in the cells by simply introducing the 3′UTR KH-binding hairpin sequence into each mRNA. Thus, the CRAS system may be used to increase soluble fractions of functional enzymes involved in the metabolic pathway, and the CLEX system is more suitable for the production of one kind of recombinant protein, such as therapeutic peptides, enzymes, or peptides.

Based on the above description, it will be understood by those skilled in the art that the present invention may be implemented in a different specific form without changing the technical spirit or essential characteristics thereof. Therefore, it should be understood that the above embodiment is not limitative, but illustrative in ail aspects. The scope of the invention is defined by the appended claims rather than by the description preceding them, and therefore all changes and modifications that fall within metes and bounds of the claims, or equivalents of such metes and bounds are therefore intended to be embraced by the claims.

### EFFECT OF THE INVENTION

A CRAS system of the present invention including 3′UTR hairpin sequence-introduced mRNA of a target protein and a fusion protein of a chaperone and an RNA-binding domain may improve solubilization of various target proteins. Further, a CLEX system of the present invention including two cistrons may improve solubilization of various target proteins.

What is claimed is:

1. An expression cassette comprising a promoter and a gene encoding a fusion protein of a chaperone and an RNA-binding domain.

2. A composition for solubilizing a target protein, the composition comprising the expression cassette of claim 1 or an expression vector including the expression cassette.

3. The composition of claim 2, wherein the chaperone is one or more selected from the group consisting of Hsp4G, Hsp60, Hsp70, Hsp90, Hsp100, and Hsp104.

4. The composition of claim 2, wherein the chaperone is one or more selected from the group consisting of DnaJ, Dnak, GroEL, GroES, HtpG, ClpA, ClpX, ClpP, and GrpE.

5. The composition of claim 2, wherein the RNA-binding domain is K Homology (KH) domain, bacteriophage MS2 coat protein, bacteriophage PP7 coat protein, sterile alpha motif (SAM), or RNA recognition motif (RRM).

6. The composition of claim 2, the chaperone of the fusion protein is fused to the N-terminus or C-terminus of the RNA-binding domain.

7. The composition of claim 2, wherein the chaperone of the fusion protein is fused to the RNA-binding domain via a linker.

8. The composition of claim 2, further comprising an expression cassette including a promoter, a gene encoding a target protein, and a gene of a hairpin structure at the 3′-terminus thereof; or an expression vector including the expression cassette.

9. The composition of claim 2, wherein the expression cassette or the expression vector further comprises a ribosome-binding site (RBS).

10. The composition of claim 8, wherein the expression cassette or the expression vector further comprises a spacer gene between the gene encoding the target protein and the gene of the hairpin structure.

11. The composition of claim 8, wherein the gene of the hairpin structure has one or more of the gene of the hairpin structure.

12. The composition of claim 11, wherein the hairpin structure comprises a spacer gene between the hairpin structure and the hairpin structure.

13. The composition of claim 8, wherein the gene of the hairpin structure comprises a sequence transcribing an RNA site to which the RNA-binding domain linked to the chaperone binds.

14. A transformant comprising an expression cassette including a promoter and a gene encoding a fusion protein of a chaperone and an RNA-binding domain, or an expression vector including the expression cassette.

15. The transformant of claim 14, wherein the transformant is selected from a plant cell, a bacterial cell, a fungal cell, a yeast cell, and an animal cell.

16. The transformant of claim 14, further comprising an expression cassette including a promoter, a gene encoding a target protein, and a gene of a hairpin structure at the 3′-terminus thereof; or an expression vector including the expression cassette.

17. A kit for producing a target protein, the kit comprising the composition of claim 2.

18. A kit for producing a target protein, the kit comprising the transformant of claim 14.

19. A method for producing a target protein, the method comprising the steps of:
(i) preparing a transformant including (a) an expression cassette including a promoter and a gene encoding a fusion protein of a chaperone and an RNA-binding domain, or an expression vector including the expression cassette and (b) an expression cassette including a promoter, a gene encoding a target protein, and a gene of a hairpin structure at the 3′-terminus thereof, or an expression vector including the expression cassette; and
(ii) culturing the transformant.

20. The method of claim 19, further comprising the step of (iii) collecting the target protein from the transformant or a culture thereof.
